SEQUENCE LISTING (2 sequences)

SEQ ID NO: 1 - DNA, 14759 bp, Homo sapien
  CDS (3639)...(3984)
  CDS (11757)...(12601)
ccttggttttggggatcatttgggcaagcccgaggtgctgtgcatgggggctcctggaatcctgggaagggcagaaagccttggccccagactcatcgtgcagcagctctgagcagtatttcggctgaggagtgacttcagtgaatattcagctgaggagtgacttggccacgtgtcacagccctacttcttgggggcctggtggaagagggtggcgtagaaggttccaaggtcccaaactggaattgtcctgtatgcttggttcacacagtgcgttattttaccttcctctgagctgctaatcgcctgcctctgagctgggtgagataaatatcacaaggcacaaagtgattgtacaataaaaaaatcaaatccctcccatccatccttcagtctgccacacacgcagtctacgttacacacatgtcacgtaaagcaggatgacatccatgtcacatacatagacatattaaccgaaatgtggcccttcggttgcatatattctcatacatgaatatatttatagaaatatatgcacatatttttgtatattggatatatttatgtaactataaatttacatgcgtatggatatgaaaataaatgcatacacatttatgtaaaaaaatttgtacacatgcatttacatatgtaaatacatacatctctatgtattaatgtttaaaaacactcaatttccagcctgctgttttcttttaattttcctcctattccggggaaacagaagcgtggatcccacgtctatgctatgccaaaatacgctgtaattgaggtgttttgttttgttttgttttttgaaatcgtatattaccgaaaaacttcaaactgaaagttgaataacgggcccagcggggaaataagaggccagaccctgaccctgcatttgtcctggatttcgcctccagagtccccgcgagggtccggcgcgccagctgatctctcctttgagagcagggagtggaggcgcgagcgccccccttggcggccgcgcgcccccgccctccgccccaccccgccgcggctgcccgggcgcgccgtccacacccctgcgcgcagctcccgcccgctcggggatccccggcgagccgcgccgcgaagggggaggtgttcggccgcggccgggagggagccggcaggcggcgtcccctttaaaagccgcgagcgccgcgccacggcgccgccgccgccgtcgccgccgccggagtcctcgccccgccgcgctgcgcccggctcgcgctgcgctagtcgctccgcttcccacaccccgccggggactggcagccgccgccgcacatctgccgccacagcctccgccggctacccgaacgttctcggggccagcgccgagtggatcaccggggaccgcgaggcacccgcgcgccgcagaccccgcgcgggctggagcacccggcagagcgcgccacagcgccgtggcctctgctgcccgggctgcgccagagccgcggacgggcgcgcagagcgccggggactccggagccgatccctagcgccgcgatgcggagcacctactgcaggagatcgggggcctgggacgcgctggccgaggtgtgatcggaccccaggctagccacaaagggcacttggccccagggctaggagagcgaggggagagcacagccacccgcctcggcggcccgggactcggctcgactcgccggagaatgcgcccgaggacgacggggcgccagagccgcggtgctttcaactggcgagcgcgaatgggggtgcactggagtaaggcagagtgatgcgggggggcaactcgcctggcaccgagatcgccgccgtgcccttccctggacccggcgtcgcccaggatggctgccccgagccatgggccgcggcggagctagcgcggagcgcccgaccctcgacccccgagtcccggagccggccccgcgcggggccacgcgtccctcgggcgctggttcctaaggaggacgacagcaccagcttctcctttctcccttcccttccctgccccgcactcctccccctgctcgctgttgttgtgtgtcagcacttggctggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtagaggacgcgggccagggcccggggtgggtggtgggtgggagggggatttgggcagccactgcggtagagcccttccttacgtccaggccagaagtaaacagacccctctccagtccacgtgcaacggagccctgcaggggctcccacttccagctgccccgggcgaccgtaagcctcaccctcccggcccgcactcttccacccctctttcttcccctctccctggaatacttttggagctgttaacacttagatgaggtgttttatttatttatttatttatttttaatttttttaaaaacttttttgggtcaaagaaatccctttgagagggtagcccctgggtttcacccgttagctgagaacctgtccgctctgccatggtgatctccattcttcaagtgtttccgggagacttggtttctttgctcagagccgtgtcccatttaggaaagtactaggagtttggggttctccctacttgtttccagaaatgcgaggggtcagtactgaaggatcacttggtactgtgtttttaacagctgacacgtgcattaatagatattcaccatttacgtaatcccgggaagatacatgtgtatcttgactgcactgtggggatgcgggatggagctgcctttcgagacacccctgagggtaggggcctgggacacaagtcataagtggcttcagaagttgtggccttgagcttacagggtctggaagctataagggtgtgtgtgtgtgtgtgtgtgtgtgtgtgtgtgtgtcaggaagttctatacagtgcctctaaggaagtcacatgcaccatttatgtgtgtttatatgccagacagcgctcagcactccgcatttgggtttgtataggggacgcagggtgtcagatcaagcggtggttttcccaggttcccggcattggctgtcagcgctgtgtcacacacaaaaaagtgacagtcattggcgctggtttggttgggggggagggcaaatcccaaatctgatgtcagacgagctaagcgttggatgggagcgataaatcatctggttcaggaacttgggacccttcattatcccaaacgtttgagcttcggtcggtcttacctagactcgtgagtgtgccaagccaggagggcatcctggaggaggcacgccagccaaatgggagaccgggccgcgggggcgcgaggggggaggactgggcggggaactcgggtgactcacgtcggtcctgtccgcaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaaggtgaggcatggagcagggcgtgggggcggggagtcaccctgcaaagccctccaccgtgggcagactgcagccgtccctgtagaggcagcttggccggggcaccagcggacgtttccactcttgcttctgtactatcgtttctgaatctgattttaactcactgcttgtgtggtgggggagccagggattcccctttagtaactccgcaccctcttcctggcttgcagccagaagagctactcctcctggaagaattggagagaaatcaagtgatggggaagatgagggcaaaaggcatgcctctagtcagctaaacgtgcaagaattccacagagggaaaaggagaaaaagggaggcagattgagatttctttaagtctgtttggaagcttttgctctataaatctgccgcttaagccagggttttagggtagacagagccaagggcagagttttcagagatagtattgaaaaatcaaagcccagggccccaaagtctttctaatttatagttgatctgggcctggtttggaagattttgaatcccaatctaatccccgtgggagatcaatactacaatcaatcttattgtttccacaatgactttcttgtcctgtgcttaaatctgagataggctctgagtagagacaaggcaagccttcagataaaagcgtttgtagcagctgcctgtttttttttcatgtgcaccgaaatgtggatttttttttcttttatgatactacatgtggtttttctaaggtgggatatttctgcttgtttcatcagaagggcatttagtggactggaaatgtcttacagcagctattgaggtctgctgtacctaagttcttagagcaattagtcaaaaatatgttccacttcaattctttttctacacttttaaatgcttctttggcttaatacatttaaaatagagcatgggtttcttcaattcctagaaaagagtacaaaagtgtatatcacagagcaaccacttggcagatatttggggagttgggagtgaagttctctttcttgcctttccctgcttaggtggtaaatttcaagtgggaaatttacactgataatagactaatgggaaatggcacttccagatgttttctcccagtgtgaagggtgacttatacttgtgagagtatttgttggtaatgggaataagtcccaaaggcaagccacatagcagaagatacgttctcattgaggcagctacacattacgacggggacactgaattgatcatcagttcatttacaagcacatttctaagtgaggtgctctctgctagcagaaatcagatttgaaaggcagtaagatctcactccactctttcagaattcatccaatgaaagcagaaatcacctgttgtcatatgtaaaatttgtgtgtatgtgtacattctgccatcttaaccctgaaatgattatagatccagctaatcattcccaggtaatgctgattagaatacttttttttttgtataggaatgtaataagaacaactgttttagacacctcttctggaaatttagcatggaagctctcaactttatttttaaggcctggaagatgctgtgtctctgttacaacttaaaaggaagatcatttaagttagttaacacctaaaacattccattgtgtgaggattttatcagtgatgtctgcatattctcatcattcatctagaagtggtttgatcagaactaaacaggctacacgttattcaactgtgttattttaacttaaaaagcatgcttgagtttataaaatcagaatttatatctttgtgagtgtaaatgttacctgagaaacagtacagaagtgaccaacttgattaaaatcaacttgtaataacttcaggtcttaatgcagttagataatggagaaaagctatgtaattttgccccaaatttcaactaatccatttcttgtctcattatgactaatatatcatccttaatctggatggatatagcacttttttcaagactaatcattgttgtatacacccaggatttgcttttgataaacatccttgtgccatgcatgccacgaaaaaagtttttggtaaaccatgtgatgaaggttgctggctcaagaacagaatttagtttctacagcattaatgagcatttatttgaaaaaagaccataaagacccaatcataagaattacctgttgggttttctttgtaggtgtgatcgaatggtttggtggaattactcgacgagatatcatgatagcattctttcaaccaatatgagtataatgcgaccatatcataggggatctgagacagaattatcagttgtatttttcctattgaattttgtctagtcctttctccagtggcttttatttgggagaatatcagctttgctaaaatgttattgttttcaagatcattaaaaagtgcttcagctacatagacctttggaaactgccattgaacatagaaaagtcagttctgcaagtggaaagagtgttttgtgtattgctgtagttggaaacacattgaaactggttgacttcactggccctccaaaaagtctttatgcttttttgtcagatgggagagagaaagaccaggtgcttcttgttctcctcactctgaaggacacagtcttctttctacatgaaataactggattatttgcctctgtgactgaagctttcaaatagagattaaccctctttccacaaatataattattatgaaaatatccatataatagaaaagttcaagaaataactattgccctgcattagagactttgtggcacaaattcccccgtgcaaacaacagatttggacacatagatccaccaaaaccaatacttacctggtatggttccctagtggccccaggtatttcattgtcattacagaggccacattaagtaggaaaattactctatttggaaatggttgttgagattgaggctttggtgtccagtgatacttccttggcactgacattttccgttccacctgttttttagtggttcccctaaatttctcttaatccctttgcagtgaactattttgcgttcttagacttgctctttgtgtattttcactgagacaataagagaatatttcatcattccgaaggtgttggtgttaagggtgggcagaggccaaatcagggttgttgatgacaaccatgctctctattcctttatttgccattcccttgttgtattttttttaaaatggaatgtttttaaccttttgtatttgatattttttttctccttgatcagttgtctgttattttattatctggaaaatcttatattatactcagcctctttcattttgtgttagggcagtgacttccagccttactgattgccagcatatccccaggttttgttgttgttgttgttgttttactggagattttttagcccaaagtgtgttttaaaatcctcgaagcataacggtaacttacttttttgataaaacttaccatactttatttagaacaaaagggcagccacaaaatagcagtggctccttataaaatagacacattccagtgggccccgtcacttttctgctcatttctgtctgttctgtccatcatacctaagtcatatatttctgttcatttagttgggacagaactcacccaatgttatcattgtactaaatataaatgtgcccctaatggttttgacttttgcttaagtttttgagtcctcatgtatgttaggtagtgccatctagtagccagaaatttgggaactggctgggcatgatggctaatacctgtaatcccagcactttgggaggcttaggtgggtggatcacttgaggtcaggagttccagaccagcctggccaacatggtgaaaccacatctctactaaaatataaaaaaatagccaggtatgatggcccatgcctgtaatccgagctaattgggaggctgagatgggaggactgcttgaacctgggaggtggaggctgctgtgagccaagattgtgccactgcactccagcctgggcaacagagtgagaccctgtgtcacaaaaacaagaaacaaaacaaaacaaaagacaagaaacctgagaagcgcagtagattcaattatatatatctacttttaatttgctagctctgtgaccttaggaaagttacataacctctctgaactgcaactgtttcatttacaaaatggagataatgatagtttttctctaattggtttgttgtgagataattcatataaagctgatggtgccagattacactcaaaaaaagcattcagctgtcattatcattatgacttcttttgttaatgttatagcctttccttctctagggaaaaggaggccagagtggacctaggctgactgagagaattcagctcagtcttttgaattattttgaggtagaggaatgattgatatagtatagattattaaattaggacttcacttttggagaaaagttcagatatcattgttgtcttatttttcttcactttcccacatttttgcagccatagctccatccatttggttaagaacttagaagctcacaaactcgggtcaaagacaggtcgaaatcctcaaatcccttaagaacttcagcttattcaggaagggatatttacagaaaactagcaattgtataagtctccaaaaaagcatacattacttgaggatccatatatttttggcatcctcagggttgctgtgatgatttatagaaggtttgtttatttaatttactttatttcaaataggttttaatttttgtacccttaagaaaagattcgtactcttccctggcagattaaagaaaatgagcgtatattccctaaccttggccagttactttcctgggtttgagggtttctgtgaacgtctaacttacctctgtgacctgtttctgcaaccaggggtgttgcaatggatgcttttgtcttgaggatgggacctttcaagaaacagattcactgaggtgcagtgggaaggtcagagaaagatcttcgtatcgcctattattatttgctcgtctattttttctcctttcttaaggccactaactgattctcctttgctaaggctgcctacttccactgagaccttgaaccacatgaaattgttgttgtctgtgtttctggtcaaatagtggcaattttgtatgattcaatcttgtcatttaattttttgggaggttattattctatttcataccttttttatacccatcttctttacttcatttacctgtccctcatacttgacttgtagcttgtcccttcactgtcatcgtctggccatgtgggtgtgtacgtgtgtgcgagagagagaatgtgtgagaatgtatgtttctttatgcattgggatttagggtttttcttgcaattgtgatttctctgggcacttttgttaatatagctagtcagcgagtgctctagataattttccttgcctccccctctttgaaagaaaagagggtgttcttagatgtattcttatcagataagccagtagctcaggtgctggtctggctttggtgtcattggggtctgaggttgctgacttttaccttctctgctgaaaaattaccttcagcagaaacgtctgaattgcaaggagaaggagaaaaaaacaggccaaacacagtccttggtactccttgggagccactgagaagagtccaggttcaaatggtcagaaggttattttaatgattgtgtctggcctaaagtaccattagcttccagtggagtttagaatgtggatggatcctgaaaggtattccccagaggtttggattaataggcacaagggaaccctaaaggactctattggcctgatactccccatatccacgtagaagagctttagaagaaccttctgttctgagaccctggctgggcccacccagagctggcccattcaactcttactcctttgccaccactaatggttcttctactagtttttatattatttaacaaaaaggcactttaaaaatgcactcctggcaatctatactggaatatgaaaaacatgctgcaaaaccttgacactccaagtgtggtcttacagttcccagaatcccctccttgaggagctgctagaaatgctgaatctcaagcatctccccagacctactgaatcagagcctgcatctgaagctttacggtgtacaagctgttttatgtgaaggctgaagtttgaaaagcactgcattaaagcgttagtttggtataaactgccctgactgaacttggtgtgtccacttagcttgcatgatgactgttgctttgatgatgaaggcttacacgggtagatcctttgagtgagtgatctgacatgattctcctttgctaaggcatctagattcagtgcacaacttacagctgtttgtctttaggggaaatacaactgtaaaattaataaaaacatagtctcttcttatgataacatggaacgatggcaaaatagattttgttagcacttgggtaggaattctgaatgaagcaggcaaattctgttggcagtgaaatgataggatgtggtaaagttagaataaaataaacttaaatgtctcaaactctcatggtatatactaccagtttaataataatgttgtacctttgatgatttgcagactacaagcattcaaggtgctgtgttatatattacttgcttggagaataatacttcttaaaaattgaaattcagaaattttaaatcagacaaagcttttgtgcatggcccacttaaatggctattttgaaataatgatagtggatatagaaggattattctgtaataggatgagactgttccttttgtcatggagatcataatcatatttttgtaaatttttattatttttttggttttgtgtccatcctgcacactattactgggtaggtacatggttttttaacatggtttatctttcaaaactataaaggcattgcaaacagaagacaggtcatttatttttcttccaaaagcatctaaaatgagattttgatatttgaggtcataaagaggtgagagaacagacaacagttgggaaagctatttctcttgaaattgtttggccttaattactacagtgtcctagtaccacccatacgtttccaaagaagtagatccctgtaaatgcctttgtctctggacttttgagtaaaatagtagggtgtgctttgcaaaatgtcatcgttgatgttgagtttcagagtctttaattaggaagctgaaatctgtatatcgagatttgtaaatcatctaaattgcagagtaatgttttagaatactgcttaagggattggcattaaagccttttttaaaaaagaaatgcaataatttcctcaaatcctcactcattagacctctactaactatagtgctgacttttttttttttttaccctaaagtctggaattccaaagaaatgcttcaccatttcccccattattatagccacctggaagcagtattcatgtattagatcaaaaacacaacaaagaattatgaaaggttgtttcctggtatgcaatgcatgatgacatgaacttacagaacagagagaagggaggctccatgtttatttaaagaggaaatttttattttctggttacctacttttacatgggttacatcaaatcccacgatgaggtttaaaaattctcatagataatcaaacgtcattacttggcttactgaaattcagacttttcttttttcttccctgtttttctctatcaaattagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatatatatatattttagaaaaaagaaaaaaacaaacaaacaaaaaaaccccaccccagttgacactttaatatttcccaatgaagactttatttatggaatggaatggaaaaaaaaacagctattttgaaaatatatttatatctacgaaaagaagttgggaaaacaaatattttaatcagagaattattccttaaagatttaaaatgtatttagttgtacattttatatgggttcaaccccagcacatgaagtataatggtcagatttattttgtatttatttactattataaccactttttaggaaaaaaatagctaatttgtatttatatgtaatcaaaagaagtatcgggtttgtacataattttccaaaaattgtagttgttttcagttgtgtgtatttaagatgaaaagtctacatggaaggttactctggcaaagtgcttagcacgtttgcttttttgcagtgctactgttgagttcacaagttcaagtccagaaaaaaaaagtggataatccactctgctgactttcaagattattatattattcaattctcaggaatgttgcagagtgattgtccaatccatgagaatttacatccttattaggtggaatatttggataagaaccagacattgctgatctattatagaaactctcctcctgccccttaatttacagaaagaataaagcaggatccatagaaataattaggaaaacgatgaacctgcaggaaagtgaatgatggtttgttgttcttctttcctaaattagtgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacacgcagcaaaattatgcacatcgtgttttctgcccaccctctgttctctgacctatcagcttgcttttctttccaaggttgtgtgtttgaacacatttctccaaatgttaaacctatttcagataataaatatcaaatctctggcatttcattctataaagtccaacctgtaagagaaaatggtgcatttgtatagcgcttacaatgatgaccttgtgtttgcatttttgtttctgaagttatatattttagagggggtgggggaaaggtaatgaatggctggaaaattgcaggcaagttatttgataagtcatatttgcactaaaggtgttaccagtgatttagtatttttcaaatgaacttctttggggcagaaagatttaagggaaaactaaagcctacaaaacaagcaaaacctggataacccgagataaagtttcagagataatagcccatgcaacagaggcaacggtgccagaaaattagaaagggaaagtgtcggagatcagcttctataagaacatctgccagttggactgacgcccaaacagaatgaagtcaaattaggctgctcagattgaacacttaccagagtgtcagggcttctgtaccctgggttagaatcagaccaaggaagggttcagcagatgttcataagagcagggcacccacaactacccactattttactggcagtattttaggtcagtttccaggactttgcatcccctctgatcctgccatgcatgattggtgaaacctacctctaatctccttggaattggctaaaaaacagtgtgtttataatggaacagactgttataatcaaattcttcctaggaattaacttttgatgactatgagcttagttacagttcggaggttatgaggttatgtaaaccttatctttaaatgtgcatgacagttatcttttactaatgctggttaacttttaaaatcttgcagctcctttttatctctagttctattgttcttgattaggtgagaaccattagatcatacccaactgaggggattggggtcttgtttgttctccagctgttcttcaccctctattgccatggacatgaaggacagactgcacggtcttaacatgttaaaacgaatgacccatgttttctcatat

SEQ ID NO: 2 - protein, 396 aa, Homo sapien
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

(19) United States
(12) Patent Application Publication
(10) Pub. No.: US 2003/0176344 A1
(43) Pub. Date: Sep. 18, 2003

(54) Human osteoporosis gene

(75) Inventors: Unnur Styrkarsdottir, Reykjavik (IS); Vala Drofn Johannsdottir, Reykjavik (IS)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: deCODE genetics ehf., Reykjavik (IS)

(21) Appl. No.: 10/346,723

(22) Filed: Jan. 16, 2003

Related U.S. Application Data
(63) Continuation-in-part of application No. 09/952,360, filed on Sep. 13, 2001.
(63) Continuation-in-part of application No. PCT/IB01/01667, filed on Sep. 12, 2001.
Application No. PCT/IB01/01667 is a continuation-in-part of application No. 09/661,887, filed on Sep. 14, 2000.

(51) Int. Cl. C12Q 1/68; C07H 21/04; C07K 14/475

(52) U.S. Cl. 514/12; 435/6; 536/23.5; 530/350

(57) ABSTRACT

A role of the human BMP2 nucleic acid in osteoporosis is disclosed. Methods for diagnosis, prediction of clinical course and treatment for osteoporosis or a susceptibility to osteoporosis using polymorphisms in the BMP2 nucleic acid, alone or in combination with other assays, are also disclosed.

### RELATED APPLICATION

This application is a continuation-in-part of U.S. application Ser. No. 09/952,360, filed Sep. 13, 2001, and which is also a continuation-in-part and claims priority to International Application No. PCT/IB01/01667, which designated the United States and was filed on Sep. 12, 2001, published in English, which is a continuation-in-part of U.S. application Ser. No. 09/661,887, filed Sep. 14, 2000. The entire teachings of the above applications are incorporated herein by reference.

### BACKGROUND OF THE INVENTION

Osteoporosis is a debilitating disease characterized by low bone mass and deterioration of bone tissue, as defined by decreased bone mineral density (BMD). A direct result of the experienced microarchitectural deterioration is susceptibility to fractures and skeletal fragility, ultimately causing high mortality, morbidity and medical expenses worldwide. Postmenopausal woman are at greater risk than others because the estrogen deficiency and corresponding decrease in bone mass experienced during menopause increase both the probability of osteoporotic fracture and the number of potential fracture sites. Yet aging women are not the only demographic group at risk. Young woman who are malnourished, ammenorrheic, or insufficiently active are at risk of inhibiting bone mass development at an early age. Furthermore, androgens play a role in the gain of bone mass during puberty, so elderly or hypogonadal men face the risk of osteoporosis if their bones were insufficiently developed.

The need to find a cure for this disease is complicated by the fact that there are many contributing factors that cause osteoporosis. Nutrition (particularly calcium, vitamin D and vitamin K intake), hormone levels, age, sex, race, body weight, activity level, and genetic factors all account for the variance seen in bone mineral density among individuals. Currently, the drugs approved to treat osteoporosis act as inhibitors of bone reabsorption, and include methods such as hormone replacement therapy (HRT), selective estrogen receptor modulators, calcitonin, and biophosphonates. However, these treatments may not individually reduce risk with consistent results and while some therapies improve BMD when co-administered, others show no improvement or even lose there efficacy when used in combination. Clearly, as life expectancy increases and health and economic concerns of osteoporosis grow, a solution for the risks associated with this late-onset disease is in great demand. Early diagnosis of the disease or predisposition to the disease would be desirable.

### SUMMARY OF THE INVENTION

As described herein, it has been discovered that polymorphisms in the nucleic acid sequence for human bone morphogenetic protein 2 (BMP2) have been correlated through human linkage studies to a number of osteoporosis phenotypes. In particular, it has been discovered that one or more single nucleotide polymorphisms within the nucleotide sequence encoding the BMP2 gene product is correlated to osteoporosis. Accordingly, this invention pertains to an isolated nucleic acid molecule containing the BMP2 nucleic acid of SEQ ID NO: 1 having at least one altered nucleotide as shown in Table 2 and to gene products encoded thereby (referred to herein as a “variant BMP2 nucleic acid or gene” or “variant BMP2 gene product”).

A number of polymorphisms have been observed in the BMP2 nucleic acid as reported in Table 2. Thus, in particular embodiments, the isolated nucleic acid molecule of the invention can have one or a combination of these nucleotide polymorphisms. These polymorphisms can be part of a group of other polymorphisms in the BMP2 nucleic acid that contributes to the presence, absence or severity of osteoporosis. In one embodiment, the nucleic acid molecule will comprise at least the polymorphism at nucleotide position 3747, and the gene products will comprise the polymorphism at nucleotide position 3747 (amino acid change serine to alanine).

The invention further provides a method for assaying a sample for the presence of a nucleic acid molecule comprising all or a portion of BMP2 in a sample, comprising contacting said sample with a second nucleic acid molecule comprising a nucleotide sequence encoding a BMP2 polypeptide (e.g., SEQ ID NO: 1 or the complement of SEQ ID NO: 1 and which comprises at least one polymorphism as shown in Table 2); a nucleotide sequence encoding SEQ ID NO: 2 which comprises at least one polymorphism as shown in Table 2, or a fragment or derivative thereof, under conditions appropriate for selective hybridization. In one embodiment, the nucleic acid molecule will comprise at least the polymorphism at nucleotide position 3747, and the gene products will comprise the polymorphism at nucleotide position 3747 (amino acid change serine to alanine). The invention additionally provides a method for assaying a sample for the level of expression of a BMP2 polypeptide, or fragment or derivative thereof, comprising detecting (directly or indirectly) the level of expression of the BMP2 polypeptide, fragment or derivative thereof.

The invention also relates to a vector comprising an isolated nucleic acid molecule of the invention operatively linked to a regulatory sequence, as well as to a recombinant host cell comprising the vector. The invention also provides a method for preparing a polypeptide encoded by an isolated nucleic acid molecule described herein (a BMP2 polypeptide), comprising culturing a recombinant host cell of the invention under conditions suitable for expression of said nucleic acid molecule.

The invention further provides an isolated polypeptide encoded by isolated nucleic acid molecules of the invention (e.g., BMP2 polypeptide), as well as fragments or derivatives thereof. In a particular embodiment, the polypeptide comprises the amino acid sequence of SEQ ID NO: 2 and comprising at least one polymorphism as shown in Table 2, and in another embodiment comprising at least the polymorphism at 3747 resulting in an amino acid change from serine to alanine. The invention also relates to an isolated polypeptide comprising an amino acid sequence that is greater than about 90 percent identical to the amino acid sequence of SEQ ID NO: 2 and comprising at least one polymorphism described herein; in another embodiment, the amino acid sequence is about 95 percent identical to the amino acid sequence of SEQ ID NO: 2.

The invention also relates to an antibody, or an antigen-binding fragment thereof, which selectively binds to a polypeptide of the invention, as well as to a method for assaying the presence of a polypeptide encoded by an isolated nucleic acid molecule of the invention in a sample, comprising contacting said sample with an antibody which specifically binds to the encoded polypeptide.

The invention further relates to methods of diagnosing osteoporosis or a predisposition to osteoporosis. The methods of diagnosing a predisposition to osteoporosis in an individual include detecting the presence of a mutation in BMP2, as well as detecting alterations in expression of an BMP2 polypeptide, such as the presence of different splicing variants of BMP2 polypeptides. The alterations in expression can be quantitative, qualitative, or both quantitative and qualitative. The methods of the invention alone or in combination with other assays, e.g., bone turnover marker assays (e.g., bone scans), allow for the accurate diagnosis of osteoporosis at or before disease onset, thus reducing or minimizing the debilitating effects of osteoporosis.

The invention additionally relates to an assay for identifying agents that alter (e.g., enhance or inhibit) the activity or expression of one or more BMP2 polypeptides. For example, a cell, cellular fraction, or solution containing an BMP2 polypeptide or a fragment or derivative thereof, can be contacted with an agent to be tested, and the level of BMP2 polypeptide expression or activity can be assessed. The activity or expression of more than one BMP2 polypeptides can be assessed concurrently (e.g., the cell, cellular fraction, or solution can contain more than one type of BMP2 polypeptide, such as different splicing variants, and the levels of the different polypeptides or splicing variants can be assessed).

In another embodiment, the invention relates to assays to identify polypeptides which interact with one or more BMP2 polypeptides. In a yeast two-hybrid system, for example, a first vector is used which includes a nucleic acid encoding a DNA binding domain and also an BMP2 polypeptide, splicing variant, or fragment or derivative thereof, and a second vector is used that includes a nucleic acid encoding a transcription activation domain and also a nucleic acid encoding a polypeptide that potentially can interact with the BMP2 polypeptide, splicing variant, or fragment or derivative thereof (e.g., a BMP2 polypeptide binding agent or receptor). Incubation of yeast containing both the first vector and the second vector under appropriate conditions allows identification of polypeptides which interact with the BMP2 polypeptide or fragment or derivative thereof, and thus can be agents which alter the activity of expression of an BMP2 polypeptide.

Agents that enhance or inhibit BMP2 polypeptide expression or activity are also included in the current invention, as are methods of altering (enhancing or inhibiting) BMP2 polypeptide expression or activity by contacting a cell containing BMP2 and/or polypeptide, or by contacting the BMP2 polypeptide, with an agent that enhances or inhibits expression or activity of BMP2 or polypeptide.

Additionally, the invention pertains to pharmaceutical compositions comprising the nucleic acids of the invention, the polypeptides of the invention, and/or the agents that alter activity of BMP2 polypeptide. The invention further pertains to methods of treating osteoporosis, by administering BMP2 therapeutic agents, such as nucleic acids of the invention, polypeptides of the invention, the agents that alter activity of BMP2 polypeptide, or compositions comprising the nucleic acids, polypeptides, and/or the agents that alter activity of BMP2 polypeptide. In another embodiment, the BMP2 therapeutic agent is the human BMP2 nucleic acid or its gene product from a healthy individual who does not have osteoporosis.

In a further embodiment, the invention is directed to a method of diagnosing a susceptibility to osteoporosis in an individual, comprising detecting a polymorphism in a human BMP2 gene of SEQ ID NO: 1, wherein the presence of a “G” at nucleotide position 122247 of AL035668; the presence of a “G” allele at position 118920 of AL035668; the presence of a “T” allele at position 167584 of AL035668; the presence of a “G” allele at position 138476 of AL035668; the presence of a “T” allele at position 167584 of AL035668; the presence of a “T” allele at TSC0428253; or the presence of the “G” allele of TSC0293456 is indicative of a susceptibility to osteoporosis, compared with an individual having a “T” at nucleotide position 122247 of AL035668; an “A” allele at position 118920 of AL035668; a “C” allele at position 167584 of AL035668; an “A” allele at position 138476 of AL035668; a “C” allele at position 167584 of AL035668; a “C” allele at TSC0428253; or the presence of the “A” allele of TSC0293456. In a particular embodiment, the polymorphism is detected in a sample from a source selected from the group consisting of: blood, serum, cells and tissue.

In another embodiment, the invention is directed to a method of diagnosing a susceptibility to osteoporosis in an individual, comprising detecting a polymorphism in a human BMP2 gene of SEQ ID NO: 1, wherein the polymorphism is selected from the group consisting of T to G at position 122247 of AL035668; A to G at position 118920 of AL035668; C to T at position 167584 of AL035668; A to G at position 138476 of AL035668; C to T at position 167584 of AL035668; C to T at TSC0428253; A to G at TSC0293456 and combinations thereof. In a particular embodiment, the polymorphism is detected in a sample from a source selected from the group consisting of: blood, serum, cells and tissue.

In another embodiment, the invention is directed to an isolated nucleic acid molecule comprising the nucleic acid having SEQ ID NO: 1 with one or more of the nucleic acid changes selected from the group consisting of: T to G at position 122247 of AL035668; C to T at position 121366 of AL035668; A to G at position 118920 of AL035668; C to T at position 167584 of AL035668; A to G at position 138476 of AL035668 and combinations thereof.

In another embodiment, the invention is directed to an isolated polypeptide comprising an amino acid sequence selected from the group consisting of: an alanine at amino acid position 37, a serine at amino acid position 94, a serine at amino acid position 189, or combinations thereof.

In yet another embodiment, the invention is directed to a method of diagnosing a susceptibility to osteoporosis, comprising detecting a polypeptide described herein that is indicative of a susceptibility to osteoporosis. In a particular embodiment, the determination of an amino acid at a position selected from the group consisting of: position 37, position 94, position 1891 and combinations thereof, comprises contacting the sample with an antibody specific for either the reference amino acid or the variant amino acid. In another embodiment, the invention is directed to a pharmaceutical composition comprising a polypeptide described herein. In another embodiment, the invention is directed to a method of treating osteoporosis in an individual, comprising administering to the individual, isolated polypeptide described herein, in a therapeutically effective amount.

In another embodiment, the invention is directed to a kit comprising: a) at least one antibody selected from the group consisting of: an antibody specific for the BMP2 protein, comprising a serine at amino acid position 37, an alanine at amino acid position 37, an alanine at amino acid position 94, a serine at amino acid position 94, an arginine at amino acid position 189, a serine at amino acid position 189, or combinations thereof; and b) a reference BMP2 protein sample.

### DETAILED DESCRIPTION OF THE INVENTION

As described herein, Applicants have completed a genome wide scan on patients with various forms of osteoporosis and identified a region on chromosome 20 linked to osteoporosis. Until now there have been no known linkage studies of osteoporosis in humans showing any connection to this region of the chromosome. Based on the linkage studies conducted, Applicants have discovered a direct relationship between BMP2 and osteoporosis. Although the BMP2 nucleic acid from normal individuals is known, there have been no studies directly investigating the link between BMP2 and osteoporosis. Moreover, there have been no variant forms reported that have been associated with osteoporosis. The linkage studies are based on genome wide scans encompassing affected persons having different osteoporosis phenotypes; i.e., hip, spine, combined and combined severe (e.g., patients having vertebral compression fracture, hip fracture, other osteoporosis related low impact fracture). From the data obtained in the linkage study, a region on chromosome 20, specifically the BMP2 gene, was identified. The variant BMP2 nucleic acid has previously unreported nucleotide changes that were observed in the patient population (see Tables 2, 3 and 4 for the polymorphic changes).

All nucleotide positions are relative to SEQ ID NO: 1 or to GenBank number AL035668, where indicated. The polymorphism at nucleotide position 3747 appears statistically more frequent in the osteoporosis test population than in the control population.

Nucleic Acids of the Invention

BMP2 Nucleotides, Portions and Variants

Accordingly, the invention pertains to an isolated nucleic acid molecule comprising the human BMP2 nucleic acid having at least one nucleotide alteration and correlated with incidence of osteoporosis. The term, “variant BMP2”, as used herein, refers to an isolated nucleic acid molecule in chromosome 20 having at least one altered nucleotide. The variants of BMP2 of the present invention are associated with a susceptibility to a number of osteoporosis phenotypes. The invention includes a portion or fragment of the isolated nucleic acid molecule containing the alteration (e.g., cDNA, the gene, or a fragment thereof), including fragments encoding a variant BMP2 polypeptide (e.g., the polypeptide having SEQ ID NO: 2). In one embodiment, the isolated nucleic acid molecules comprises a polymorphism selected from the group consisting of any one or combination of those shown in Tables 2, 3 and 4 of the BMP2 gene, including comprising at least the polymorphism at nucleotide 3747. In certain embodiments for therapeutic purposes for example, the nucleic acid comprises the sequence of SEQ ID NO: 1 that represents the human BMP2 nucleic acid of a healthy individual.

The isolated nucleic acid molecules of the present invention can be RNA, for example, mRNA, or DNA, such as cDNA and genomic DNA. DNA molecules can be double-stranded or single-stranded; single-stranded RNA or DNA can be either the coding, or “sense” strand or the non-coding, or “antisense” strand. The nucleic acid molecule can include all or a portion of the coding sequence of the gene and can further comprise additional non-coding sequences such as introns and non-coding 3′ and 5′ sequences (including regulatory sequences and other flanking sequences, for example). Additionally, the nucleic acid molecule can be fused to a marker sequence, affinity tag, or other gene or sequence, for example, a sequence that encodes a polypeptide to assist in isolation or purification of the polypeptide. Such sequences include, but are not limited to, those that encode a glutathione-S-transferase (GST) fusion protein and those that encode a hemagglutin A (HA) polypeptide marker from influenza.

An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography such as HPLC. An isolated nucleic acid molecule of the invention can comprise at least about 50, 80 or 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Therefore, recombinant DNA contained in a vector are included in the definition of “isolated” as used herein. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The present invention also pertains to nucleic acid molecules that are not necessarily found in nature but, nonetheless, encode a BMP2 polypeptide (e.g., a polypeptide having the amino acid sequence of SEQ ID NO: 2 and comprising at least one polymorphism as shown in Tables 2, 3 and 4). Thus, for example, DNA molecules that comprise a sequence that is different from the naturally-occurring nucleotide sequence but that, due to the degeneracy of the genetic code, encode an BMP2 polypeptide of the present invention are also the subject of this invention. The invention also encompasses variants of the nucleotide sequences of the invention, such as those encoding portions, analogues or derivatives of the BMP2 polypeptide. Such variants can be naturally-occurring, such as in the case of allelic variation, or non-naturally-occurring, such as those induced by various mutagens and mutagenic processes. Intended variations include, but are not limited to, addition, deletion and substitution of one or more nucleotides, which can result in conservative or non-conservative amino acid changes, including additions and deletions. The nucleotide (and/or resultant amino acid) changes can be silent or conserved; that is, they do not necessarily have to alter the characteristics or activity of the BMP2 polypeptide. In one embodiment, the nucleotide sequences are fragments that comprise one or more polymorphic microsatellite markers. In another embodiment, the nucleotide sequences are fragments that comprise one or more single nucleotide polymorphisms in the BMP2 gene.

Other alterations of the nucleic acid molecules of the invention can include, for example, labeling, methylation, internucleotide modifications such as uncharged linkages (e.g., methyl phosphonates, phosphotriesters, phosphoamidates, carbamates), charged linkages (e.g., phosphorothioates, phosphorodithioates), pendent moieties (e.g., polypeptides), intercalators (e.g., acridine, psoralen), chelators, alkylators, and modified linkages (e.g., alpha anomeric nucleic acids). Also included are synthetic molecules that mimic nucleic acid molecules in the ability to bind to a designated sequences via hydrogen bonding and other chemical interactions. Such molecules include, for example, those in which peptide linkages substitute for phosphate linkages in the backbone of the molecule.

The invention also pertains to nucleic acid molecules that hybridize under high stringency hybridization conditions, such as for selective hybridization, to a nucleotide sequence described herein (e.g., nucleic acid molecules which specifically hybridize to a nucleotide sequence encoding polypeptides described herein, and, optionally, have an activity of the polypeptide). In one embodiment, the invention includes variants described herein that hybridize under high stringency hybridization and wash conditions (e.g., for selective hybridization) to a nucleotide sequence comprising a nucleotide sequence selected from SEQ ID NO: 1 comprising at least one polymorphism as shown in Tables 2, 3 and 4 or the complement thereof, or a nucleotide sequence encoding an amino acid sequence of SEQ ID NO: 2 comprising at least one polymorphism as shown in Tables 2, 3 and 4. In one embodiment, the variant that hybridizes under high stringency hybridizations has an activity of BMP2.

Such nucleic acid molecules can be detected and/or isolated by allele- or sequence-specific hybridization (e.g., under high stringency conditions). “Specific hybridization,” as used herein, refers to the ability of a first nucleic acid to hybridize to a second nucleic acid in a manner such that the first nucleic acid does not hybridize to any nucleic acid other than to the second nucleic acid (e.g., when the first nucleic acid has a higher complementarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, that permit hybridization of a particular nucleic acid to a second nucleic acid; the first nucleic acid can be perfectly (i.e., 100%) complementary to the second, or the first and second can share some degree of complementarity that is less than perfect (e.g., 70%, 75%, 85%, 90%, 95%). For example, certain high stringency conditions can be used to distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules. Typically, conditions are used such that sequences at least about 60%, at least about 70%, at least about 80%, at least about 90% or at least about 95% or more identical to each other remain hybridized to one another. By varying hybridization conditions from a level of stringency at which no hybridization occurs to a level at which hybridization is first observed, conditions that will allow a given sequence to hybridize (e.g., selectively) with the most complementary sequences in the sample can be determined.

Exemplary conditions are described in Krause, M. H. and S. A. Aaronson, Methods in Enzymology, 200:546-556 (1991). Also, in, Ausubel, et al., “Current Protocols in Molecular Biology”, John Wiley & Sons, (1998), that describe the determination of wash conditions for moderate or low stringency conditions. Washing is the step in which conditions are usually set so as to determine a minimum level of complementarity of the hybrids. Generally, starting from the lowest temperature at which only homologous hybridization occurs, each ° C. by which the final wash temperature is reduced (holding SSC concentration constant) allows an increase by 1% in the maximum mismatch percentage among the sequences that hybridize. Generally, doubling the concentration of SSC results in an increase in Tₘ of about 17° C. Using these guidelines, the wash temperature can be determined empirically for high, moderate or low stringency, depending on the level of mismatch sought.

For example, a low stringency wash can comprise washing in a solution containing 0.2×SSC/0.1% SDS for 10 min at room temperature; a moderate stringency wash can comprise washing in a pre-warmed solution (42° C.) solution containing 0.2×SSC/0.1% SDS for 15 min at 42° C.; and a high stringency wash can comprise washing in pre-warmed (68° C.) solution containing 0.1×SSC/0.1% SDS for 15 min at 68° C. Furthermore, washes can be performed repeatedly or sequentially to obtain a desired result as known in the art. Equivalent conditions can be determined by varying one or more of the parameters given as an example, as known in the art, while maintaining a similar degree of complementarity between the target nucleic acid molecule and the primer or probe used (e.g., the sequence to be hybridized).

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=# of identical positions/total # of positions×100). In certain embodiments, the length of a sequence aligned for comparison purposes is at least 30%, at least 40%, at least 60%, at least 70%, at least 80% or at least 90% of the length of the reference sequence. The actual comparison of the two sequences can be accomplished by well-known methods, for example, using a mathematical algorithm. A non-limiting example of such a mathematical algorithm is described in Karlin et al, Proc. NatL. Acad. Sci. USA, 90:5873-5877 (1993). Such an algorithm is incorporated into the NBLAST and XBLAST programs (version 2.0) as described in Altschul et al., Nucleic Acids Res., 25:389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20).

Another non-limiting example of a mathematical algorithm utilized for the comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) PNAS, 85:2444-8.

In another embodiment, the percent identity between two amino acid sequences can be accomplished using the GAP program in the GCG software package (Accelrys, Cambridge, UK) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package, using a gap weight of 50 and a length weight of 3.

The present invention also provides isolated nucleic acid molecules that contain a fragment or portion that hybridizes under highly stringent conditions to a nucleotide sequence comprising a nucleotide sequence selected from SEQ ID NO: 1 and comprising at least one polymorphism as shown in Tables 2, 3 and 4 and the complement thereof and also provides isolated nucleic acid molecules that contain a fragment or portion that hybridizes under highly stringent conditions to a nucleotide sequence encoding an amino acid sequence selected from SEQ ID NO: 2, a polymorphic variant thereof, or a fragment or portion thereof. The nucleic acid fragments of the invention are at least about 15, at least about 18, 20, 23 or 25 nucleotides, and can be 30, 40, 50, 100, 200 or more nucleotides in length. Longer fragments, for example, 30 or more nucleotides in length, which encode antigenic polypeptides described herein, are particularly useful, such as for the generation of antibodies as described below.

Probes and Primers

In a related aspect, the nucleic acid fragments of the invention are used as probes or primers in assays such as those described herein. “Probes” or “primers” are oligonucleotides that hybridize in a base-specific manner to a complementary strand of nucleic acid molecules. In addition to DNA and RNA, such probes and primers include polypeptide nucleic acids (PNA), as described in Nielsen et al., Science, 254, 1497-1500 (1991).

A probe or primer comprises a region of nucleotide sequence that hybridizes to at least about 15, typically about 20-25, and in certain embodiments about 40, 50 or 75, consecutive nucleotides of a nucleic acid molecule comprising a contiguous nucleotide sequence selected from: SEQ ID NO: 1 and comprising at least one polymorphism as shown in Tables 2, 3 and 4 and the complement thereof, or a sequence encoding an amino acid sequence selected from SEQ ID NO: 2 or polymorphic variant thereof. In particular embodiments, a probe or primer can comprise 100 or fewer nucleotides; for example, in certain embodiments from 6 to 50 nucleotides, or for example from 12 to 30 nucleotides. In other embodiments, the probe or primer is at least 70% identical to the contiguous nucleotide sequence or to the complement of the contiguous nucleotide sequence, for example at least 80% identical in certain embodiments, at least 85% identical in other embodiments, at least 90% identical, and in other embodiments at least 95% identical, or even capable of selectively hybridizing to the contiguous nucleotide sequence or to the complement of the contiguous nucleotide sequence. Often, the probe or primer further comprises a label, e.g., radioisotope, fluorescent compound, enzyme, or enzyme co-factor.

The nucleic acid molecules of the invention such as those described above can be identified and isolated using standard molecular biology techniques and the sequence information provided in SEQ ID NO: 1. For example, nucleic acid molecules can be amplified and isolated by the polymerase chain reaction using synthetic oligonucleotide primers designed based on one or more of the sequences provided in SEQ ID NO: 1 (and optionally comprising at least one polymorphism as shown in Tables 2, 3 and 4) and/or the complement thereof. See generally PCR Technology: Principles and Applications for DNA Amplification (ed. H. A. Erlich, Freeman Press, NY, N.Y., 1992); PCR Protocols: A Guide to Methods and Applications (Eds. innis, et al., Academic Press, San Diego, Calif., 1990); Mattila et al., Nucleic Acids Res., 19:4967 (1991); Eckert et al., PCR Methods and Applications, 1:17 (1991); PCR (eds. McPherson et al., IRL Press, Oxford); and U.S. Pat. No. 4,683,202. The nucleic acid molecules can be amplified using cDNA, mRNA or genomic DNA as a template, cloned into an appropriate vector and characterized by DNA sequence analysis.

Other suitable amplification methods include the ligase chain reaction (LCR) (see Wu and Wallace, Genomics, 4:560 (1989), Landegren et al., Science, 241:1077 (1988), transcription amplification (Kwoh et al., Proc. Natl. Acad. Sci. USA, 86:1173 (1989)), and self-sustained sequence replication (Guatelli et al., Proc. Nat. Acad. Sci. USA, 87:1874 (1990)) and nucleic acid based sequence amplification (NASBA). The latter two amplification methods involve isothermal reactions based on isothermal transcription, which produce both single-stranded RNA (ssRNA) and double-stranded DNA (dsDNA) as the amplification products in a ratio of about 30 and 100 to 1, respectively.

The amplified DNA can be labeled, for example radiolabeled, and used as a probe for screening a cDNA library derived from human cells. The cDNA can be derived from mRNA and contained in zap express (Stratagene, La Jolla, Calif.), ZIPLOX (Gibco BRL, Gaithesburg, Md.) or other suitable vector. Corresponding clones can be isolated, DNA can obtained following in vivo excision, and the cloned insert can be sequenced in either or both orientations by art recognized methods to identify the correct reading frame encoding a polypeptide of the appropriate molecular weight. For example, the direct analysis of the nucleotide sequence of nucleic acid molecules of the present invention can be accomplished using well known methods that are commercially available. See, for example, Sambrook et al., Molecular Cloning, A Laboratory Manual (2nd Ed., CSHP, New York 1989); Zyskind et al., Recombinant DNA Laboratory Manual, (Acad. Press, 1988)). Additionally, fluorescence methods are also available for analyzing nucleic acids (Chen, et al., Genome Res. 9, 492 (1999)) and polypeptides. Using these or similar methods, the polypeptide and the DNA encoding the polypeptide can be isolated, sequenced and further characterized.

Antisense nucleic acid molecules of the invention can be designed using the nucleotide sequences of SEQ ID NO: 1 and comprising at least one polymorphism as shown in Tables 2, 3 and 4 and/or the complement thereof, and/or a portion of SEQ ID NO: 1 and comprising at least one polymorphism as shown in Tables 2, 3 and 4 or the complement thereof, and constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid molecule can be produced biologically using an expression vector into which a nucleic acid molecule has been subcloned in an antisense orientation (i.e., RNA transcribed from the inserted nucleic acid molecule will be of an antisense orientation to a target nucleic acid of interest).

In general, the isolated nucleic acid sequences of the invention can be used as molecular weight markers on Southern gels, and as chromosome markers that are labeled to map related gene positions. The nucleic acid sequences can also be used to compare with endogenous DNA sequences in patients to identify genetic disorders (e.g., a predisposition for or susceptibility to osteoporosis), and as probes, such as to hybridize and discover related DNA sequences or to subtract out known sequences from a sample. The nucleic acid sequences can further be used to derive primers for genetic fingerprinting, to raise anti-polypeptide antibodies using immunization techniques, and as an antigen to raise anti-DNA antibodies or elicit immune responses. Portions or fragments of the nucleotide sequences identified herein (and the corresponding complete gene sequences) can be used in numerous ways as polynucleotide reagents. For example, these sequences can be used to: (i) map their respective genes on a chromosome; and thus locate gene regions associated with genetic disease; (ii) identify an individual from a minute biological sample (tissue typing); and (iii) aid in forensic identification of a biological sample. Additionally, the nucleotide sequences of the invention can be used to identify and express recombinant polypeptides for analysis, characterization or therapeutic use, or as markers for tissues in which the corresponding polypeptide is expressed, either constitutively, during tissue differentiation, or in diseased states. The nucleic acid sequences can additionally be used as reagents in the screening and/or diagnostic assays described herein, and can also be included as components of kits (e.g., reagent kits) for use in the screening and/or diagnostic assays described herein.

Vectors

Another aspect of the invention pertains to nucleic acid constructs containing a nucleic acid molecule selected from the group consisting of SEQ ID NO: 1 and comprising at least one polymorphism as shown in Tables 2, 3 and 4 and the complement or a portion thereof. Yet another aspect of the invention pertains to nucleic acid constructs containing a nucleic acid molecule encoding the amino acid sequence of SEQ ID NO: 2 or polymorphic variant thereof. The constructs comprise a vector (e.g., an expression vector) into which a sequence of the invention has been inserted in a sense or antisense orientation. As used herein, the term “vector” refers to a nucleic acid molecule capable of transporting another nucleic acid to which it has been linked. One type of vector is a “plasmid”, which refers to a circular double-stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, called expression vectors, are capable of directing the expression of genes to which they are operably linked. In general, expression vectors of utility in recombinant DNA techniques are often in the form of plasmids. However, the invention is intended to include such other forms of expression vectors, such as viral vectors (e.g., replication defective retroviruses, adenoviruses and adeno-associated viruses) that serve equivalent functions.

Recombinant expression vectors of the invention can comprise a nucleic acid molecule of the invention in a form suitable for expression of the nucleic acid molecule in a host cell. This means that the recombinant expression vectors include one or more regulatory sequences, selected on the basis of the host cells to be used for expression, which is operably linked to the nucleic acid sequence to be expressed. Within a recombinant expression vector, “operably or operatively linked” is intended to mean that the nucleotide sequence of interest is linked to the regulatory sequence(s) in a manner that allows for expression of the nucleotide sequence (e.g., in an in vitro transcription/translation system or in a host cell where the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed and the level of expression of polypeptide desired. The expression vectors of the invention can be introduced into host cells to thereby produce polypeptides, including fusion polypeptides, encoded by nucleic acid molecules as described herein.

The recombinant expression vectors of the invention can be designed for expression of a polypeptide of the invention in prokaryotic or eukaryotic cells, e.g., bacterial cells such as E. coli, insect cells (e.g., using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications can occur in succeeding generations due to either mutation or environmental influences, such progeny might not, in fact, be identical to the parent cell, but are still included within the scope of the term as used herein.

A host cell can be any prokaryotic or eukaryotic cell. For example, a nucleic acid molecule of the invention can be expressed in bacterial cells (e.g., E. coli), insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing a foreign nucleic acid molecule (e.g., DNA) into a host cell, including calcium phosphate or calcium chloride co-precipitation, DEAE-dextran mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells can integrate the foreign DNA into their genome. In order to identify and select these integrants, a gene that encodes a selectable marker (e.g., for resistance to antibiotics) is generally introduced into the host cells along with the gene of interest. Selectable markers can include those that confer resistance to drugs such as G418, hygromycin and methotrexate. Nucleic acid molecules encoding a selectable marker can be introduced into a host cell on the same vector as the nucleic acid molecule of the invention or can be introduced on a separate vector. Cells stably transfected with the introduced nucleic acid molecule can be identified by drug selection (e.g., cells that have incorporated the selectable marker gene will survive, while the other cells die).

A host cell of the invention, such as a prokaryotic or eukaryotic host cell in culture, can be used to produce (i.e., express) a polypeptide of the invention. Accordingly, the invention further provides methods for producing a polypeptide using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a nucleic acid molecule of the invention has been introduced (e.g., an exogenous BMP2 gene, or an exogenous nucleic acid encoding BMP2 polypeptide). Such host cells can then be used to create non-human transgenic animals in which exogenous nucleotide sequences have been introduced into the genome or homologous recombinant animals in which endogenous nucleotide sequences have been altered. Such animals are useful for studying the function and/or activity of the nucleotide sequence and polypeptide encoded by the sequence and for identifying and/or evaluating modulators of their activity. As used herein, a “transgenic animal” is a non-human animal, e.g., a mammal, e.g., a rodent such as a rat or mouse, in which one or more of the cells of the animal includes a transgene. Other examples of transgenic animals include non-human primates, sheep, dogs, cows, pigs, goats, chickens and amphibians. A transgene is exogenous DNA that is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, e.g., a mammal, e.g., a mouse, in which an endogenous gene has been altered by homologous recombination between the endogenous gene and an exogenous DNA molecule introduced into a cell of the animal, e.g., an embryonic cell of the animal, prior to development of the animal.

Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo (Cold Spring Harbor Laboratory Press, Cold Spring Harbor, N.Y., 1986). Methods for constructing homologous recombination vectors and homologous recombinant animals are described further in Bradley (1991) Current Opinion in Bio/Technology, 2:823-829 and in PCT Publication Nos. WO 90/11354, WO 91/01140, WO 92/0968, and WO 93/04169. Clones of the non-human transgenic animals described herein can also be produced according to the methods described in Wilmut et al. (1997) Nature, 385:810-813 and PCT Publication Nos. WO 97/07668 and WO 97/07669.

Polypeptides of the Invention

The present invention also pertains to isolated BMP2 polypeptides, e.g., proteins, and variants thereof, as well as polypeptides encoded by nucleotide sequences described herein (e.g., other splicing variants). The term “polypeptide” refers to a polymer of amino acids, and not to a specific length; thus, peptides, oligopeptides and proteins are included within the definition of a polypeptide. As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell and still be “isolated” or “purified.”

The polypeptides of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

When a polypeptide is recombinantly produced, it can also be substantially free of culture medium, i.e., culture medium represents less than about 20%, less than about 10%, or less than about 5% of the volume of the polypeptide preparation. The language “substantially free of chemical precursors or other chemicals” includes preparations of the polypeptide in which it is separated from chemical precursors or other chemicals that are involved in its synthesis. In one embodiment, the language “substantially free of chemical precursors or other chemicals” includes preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, a polypeptide comprises an amino acid sequence encoded by a nucleic acid molecule comprising a nucleotide sequence selected from the group consisting of SEQ ID NO: 1 and comprising at least one polymorphism as shown in Tables 2, 3 and 4 and complements and portions thereof. However, the invention also encompasses sequence variants. Variants include a substantially homologous polypeptide encoded at the same genetic locus in an organism, i.e., an allelic variant, as well as other splicing variants. Variants also encompass polypeptides derived from other genetic loci in an organism, but having substantial homology to a polypeptide encoded by a nucleic acid molecule comprising a nucleotide sequence selected from the group consisting of SEQ ID NO: 1 and comprising at least one polymorphism as shown in Tables 2, 3 and 4 and complements and portions thereof or having substantial homology to a polypeptide encoded by a nucleic acid molecule comprising a nucleotide sequence selected from the group consisting of nucleotide sequences encoding SEQ ID NO: 2 or polymorphic variants thereof. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods.

As used herein, two polypeptides (or a region of the polypeptides) are substantially homologous or identical when the amino acid sequences are at least about 45-55%, in certain embodiments at least about 70-75%, in other embodiments at least about 80-85%, and in other embodiments greater than about 90% or more homologous or identical. A substantially homologous amino acid sequence, according to the present invention, will be encoded by a nucleic acid molecule hybridizing to SEQ ID NO: 1 and comprising at least one polymorphism as shown in Tables 2, 3 and 4, or portion thereof, under stringent conditions as more particularly described above or will be encoded by a nucleic acid molecule hybridizing to a nucleic acid sequence encoding SEQ ID NO: 2 portion thereof or polymorphic variant thereof, under stringent conditions as more particularly described thereof.

The invention also encompasses polypeptides having a lower degree of identity but having sufficient similarity so as to perform one or more of the same functions performed by a polypeptide encoded by a nucleic acid molecule of the invention. Similarity is determined by conserved amino acid substitution or by structural similarity. Such conservative substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe and Tyr. Guidance concerning which amino acid changes are likely to be phenotypically silent are found in Bowie et al., Science 247:1306-1310 (1990).

A variant polypeptide can differ in amino acid sequence by one or more substitutions, deletions, insertions, inversions, fusions, and truncations or a combination of any of these. Further, variant polypeptides can be fully functional or can lack function in one or more activities. Fully functional variants typically contain only conservative variation or variation in non-critical residues or in non-critical regions. Functional variants can also contain substitution of similar amino acids that result in no change or an insignificant change in function. Alternatively, such substitutions can positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis (Cunningham et al., Science, 244:1081-1085 (1989)). The latter procedure introduces single alanine mutations at every residue in the molecule. The resulting mutant molecules are then tested for biological activity in vitro. Sites that are critical for polypeptide activity can also be determined by structural analysis, for example, by crystallization, nuclear magnetic resonance or photoaffinity labeling (Smith et al., J. Mol. Biol., 224:899-904 (1992); de Vos et al. Science, 255:306-312 (1992)).

The invention also includes polypeptide fragments of the polypeptides of the invention. Fragments can be derived from a polypeptide encoded by a nucleic acid molecule comprising SEQ ID NO: 1 and comprising at least one polymorphism as shown in Tables 2, 3 and 4 or a portion thereof and the complements thereof. However, the invention also encompasses fragments of the variants of the polypeptides described herein. As used herein, a fragment comprises at least 6 contiguous amino acids. Useful fragments include those that retain one or more of the biological activities of the polypeptide as well as fragments that can be used as an immunogen to generate polypeptide-specific antibodies.

Biologically active fragments (peptides which are, for example, 6, 9, 12, 15, 16, 20, 30, 35, 36, 37, 38, 39, 40, 50, 100 or more amino acids in length) can comprise a domain, segment, or motif that has been identified by analysis of the polypeptide sequence using well-known methods, e.g., signal peptides, extracellular domains, one or more transmembrane segments or loops, ligand binding regions, zinc finger domains, DNA binding domains, acylation sites, glycosylation sites, or phosphorylation sites.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the polypeptide fragment and an additional region fused to the carboxyl terminus of the fragment.

The invention thus provides chimeric or fusion polypeptides. These comprise a polypeptide of the invention operatively linked to a heterologous protein or polypeptide having an amino acid sequence not substantially homologous to the polypeptide. “Operatively linked” indicates that the polypeptide and the heterologous protein are fused in-frame. The heterologous protein can be fused to the N-terminus or C-terminus of the polypeptide. In one embodiment the fusion polypeptide does not affect function of the polypeptide per se. For example, the fusion polypeptide can be a GST-fusion polypeptide in which the polypeptide sequences are fused to the C-terminus of the GST sequences. Other types of fusion polypeptides include, but are not limited to, enzymatic fusion polypeptides, for example β-galactosidase fusions, yeast two-hybrid GAL fusions, poly-His fusions and Ig fusions. Such fusion polypeptides, particularly poly-His fusions, can facilitate the purification of the recombinant polypeptide. In certain host cells (e.g., mammalian host cells), expression and/or secretion of a polypeptide can be increased by using a heterologous signal sequence. Therefore, in another embodiment, the fusion polypeptide contains a heterologous signal sequence at its N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists. Bennett et al., J. Mol. Rec., 8:52-58 (1995) and Johanson et al., J. Biol. Chem., 270,16:9459-9471 (1995). Thus, this invention also encompasses soluble fusion polypeptides containing a polypeptide of the invention and various portions of the constant regions of heavy or light chains of immunoglobulins of various subclass (IgG, IgM, IgA, IgE).

A chimeric or fusion polypeptide can be produced by standard recombinant DNA techniques. For example, DNA fragments coding for the different polypeptide sequences are ligated together in-frame in accordance with conventional techniques. In another embodiment, the fusion gene can be synthesized by conventional techniques including automated DNA synthesizers. Alternatively, PCR amplification of nucleic acid fragments can be carried out using anchor primers that give rise to complementary overhangs between two consecutive nucleic acid fragments that can be subsequently annealed and re-amplified to generate a chimeric nucleic acid sequence (see Ausubel et al., Current Protocols in Molecular Biology, 1992). Moreover, many expression vectors are commercially available that already encode a fusion moiety (e.g., a GST protein). A nucleic acid molecule encoding a polypeptide of the invention can be cloned into such an expression vector such that the fusion moiety is linked in-frame to the polypeptide.

The isolated polypeptide can be purified from cells that naturally express it, purified from cells that have been altered to express it (recombinant), or synthesized using known protein synthesis methods. In one embodiment, the polypeptide is produced by recombinant DNA techniques. For example, a nucleic acid molecule encoding the polypeptide is cloned into an expression vector, the expression vector introduced into a host cell and the polypeptide expressed in the host cell. The polypeptide can then be isolated from the cells by an appropriate purification scheme using standard protein purification techniques.

In general, polypeptides of the present invention can be used as a molecular weight marker on SDS-PAGE gels or on molecular sieve gel filtration columns using art-recognized methods. The polypeptides of the present invention can be used to raise antibodies or to elicit an immune response. The polypeptides can also be used as a reagent, e.g., a labeled reagent, in assays to quantitatively determine levels of the polypeptide or a molecule to which it binds (e.g., a receptor or a ligand) in biological fluids. The polypeptides can also be used as markers for cells or tissues in which the corresponding polypeptide is preferentially expressed, either constitutively, during tissue differentiation, or in a diseased state. The polypeptides can be used to isolate a corresponding binding partner, e.g., receptor or ligand, such as, for example, in an interaction trap assay, and to screen for peptide or small molecule antagonists or agonists of the binding interaction.

Antibodies of the Invention

Polyclonal and/or monoclonal antibodies that specifically bind one form of the gene product but not to the other form of the gene product are also provided. Antibodies are also provided that bind a portion of either the variant or the reference gene product that contains the polymorphic site or sites. The invention provides antibodies to the polypeptides and polypeptide fragments of the invention, e.g., having an amino acid sequence encoded by SEQ ID NO: 2 and comprising at least one polymorphism as shown in Tables 2, 3 and 4, or a portion thereof, or having an amino acid sequence encoded by a nucleic acid molecule comprising all or a portion of SEQ ID NO: 1 and comprising at least one polymorphism as shown in Tables 2, 3 and 4. The term “antibody” as used herein refers to immunoglobulin molecules and immunologically active portions of immunoglobulin molecules, i.e., molecules that contain an antigen binding site that specifically binds an antigen. A molecule that specifically binds to a polypeptide of the invention is a molecule that binds to that polypeptide or a fragment thereof, but does not substantially bind other molecules in a sample, e.g., a biological sample that naturally contains the polypeptide. Examples of immunologically active portions of immunoglobulin molecules include F(ab) and F(ab′)₂ fragments that can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or fragment thereof. The antibody titer in the immunized subject can be monitored over time by standard techniques, such as with an enzyme linked immunosorbent assay (ELISA) using an immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature, 256:495-497, the human B cell hybridoma technique (Kozbor et al. (1983) Immunol. Today, 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Briefly, an immortal cell (typically a myeloma) is fused to a lymphocyte (typically a splenocyte) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cells can be applied for the purpose of generating a monoclonal antibody to a polypeptide of the invention (see, e.g., Current Protocols in Immunology, supra; Galfre et al. (1977) Nature, 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology, 9:1370-1372; Hay et al. (1992) Hum. Antibod. Hybridomas, 3:81-85; Huse et al. (1989) Science, 246:1275-1281; Griffiths et al. (1993) EMBO J., 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptides from cells and of recombinantly produced polypeptides expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, ³²P, ³³P, ¹⁴C or ³H.

Diagnostic and Screening Assays of the Invention

The present invention also pertains to a method of diagnosing or aiding in the diagnosis of osteoporosis or a susceptibility to osteoporosis associated with the presence of the BMP2 nucleic acid or gene product in an individual. Diagnostic assays can be designed for assessing BMP2 gene expression, or for assessing activity of BMP2 polypeptides of the invention. Such assays can be used alone or in combination with other assays, e.g., bone turnover marker assays (e.g., bone scans). In one embodiment, the assays are used in the context of a biological sample (e.g., blood, serum, cells, tissue, synovial fluid) to thereby determine whether an individual is afflicted with osteoporosis, or is at risk for (has a predisposition for or a susceptibility to) developing osteoporosis. The invention also provides for prognostic (or predictive) assays for determining whether an individual is susceptible to developing osteoporosis. For example, alterations in the gene can be assayed in a biological sample. Such assays can be used for prognostic or predictive purpose to thereby prophylactically treat an individual prior to the onset of symptoms associated with osteoporosis. Another aspect of the invention pertains to assays for monitoring the influence of agents (e.g., drugs, compounds or other agents) on the gene expression or activity of polypeptides of the invention, as well as to assays for identifying agents that bind to BMP2 polypeptides. These and other assays and agents are described in further detail in the following sections.

Diagnostic Assays

The nucleic acids, polypeptides and antibodies described herein can be used in methods of diagnosis of osteoporosis or a susceptibility to osteoporosis, as well as in kits useful for diagnosis of osteoporosis or a susceptibility to osteoporosis.

In one embodiment of the invention, diagnosis of a susceptibility to osteoporosis is made by detecting a polymorphism in BMP2 as described herein. The polymorphism can be a change in BMP2, such as the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of the gene; duplication of all or a part of the gene; transposition of all or a part of the gene; or rearrangement of all or a part of the gene. More than one such change can be present in a single gene. Such sequence changes alter the polypeptide encoded by a BMP2 nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a susceptibility to osteoporosis can be a synonymous change in one or more nucleotides (i.e., a change that does not result in a change in the polypeptide encoded by an BMP2 gene). Such a polymorphism can, for example, alter splicing sites, affect the stability or transport of mRNA, or otherwise affect the transcription or translation of the gene. A BMP2 nucleic that has any of the alterations described above is referred to herein as a “variant nucleic acid” or, sometimes, “mutant gene.”

In a first method of diagnosing a susceptibility to osteoporosis, hybridization methods, such as Southern analysis, Northern analysis, or in situ hybridizations, can be used (see Current Protocols in Molecular Biology, Ausubel, F. et al., eds., John Wiley & Sons, including all supplements through 1999). For example, a biological sample from a test subject (a “test sample”) of genomic DNA, RNA, or cDNA, is obtained from an individual suspected of having, being susceptible to or predisposed for, or carrying a defect for, osteoporosis (the “test individual”). The individual can be an adult, child, or fetus. The test sample can be from any source that contains genomic DNA, such as a blood sample, sample of amniotic fluid, sample of cerebrospinal fluid, sample of synovial fluid, or tissue sample from skin, muscle, buccal or conjunctival mucosa, placenta, gastrointestinal tract or other organs. A test sample of DNA from fetal cells or tissue can be obtained by appropriate methods, such as by amniocentesis or chorionic villus sampling. The DNA, RNA, or cDNA sample is then examined to determine whether a polymorphism in BMP2 is present. The presence of the polymorphism can be indicated by hybridization of the gene in the genomic DNA, RNA, or cDNA to a nucleic acid probe. A “nucleic acid probe”, as used herein, can be a DNA probe or an RNA probe; the nucleic acid probe contains at least one polymorphism in BMP2. The probe can be any of the nucleic acid molecules described above (e.g., the gene or nucleic acid, a fragment, a vector comprising the gene, etc.).

To diagnose a susceptibility to osteoporosis, a hybridization sample is formed by contacting the test sample containing BMP2, with at least one nucleic acid probe. A non-limiting example of a probe for detecting mRNA or genomic DNA is a labeled nucleic acid probe capable of hybridizing to mRNA or genomic DNA sequences described herein. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate mRNA or genomic DNA. For example, the nucleic acid probe can be all or a portion of SEQ ID NO: 1 and optionally comprising at least one polymorphism as shown in Tables 2, 3 and 4, or the complement or a portion thereof. Other suitable probes for use in the diagnostic assays of the invention are described herein.

The hybridization sample is maintained under conditions which are sufficient to allow specific hybridization of the nucleic acid probe to BMP2. “Specific hybridization”, as used herein, indicates exact hybridization (e.g., with no mismatches). Specific hybridization can be performed under high stringency conditions or moderate stringency conditions, for example, as described above. In one embodiment, the hybridization conditions for specific hybridization are high stringency.

Specific hybridization, if present, is then detected using standard methods. If specific hybridization occurs between the nucleic acid probe and BMP2 in the test sample, then BMP2 has the polymorphism that is present in the nucleic acid probe. More than one nucleic acid probe can also be used concurrently in this method. Specific hybridization of any one of the nucleic acid probes is indicative of a polymorphism in BMP2, and is therefore diagnostic for a susceptibility to osteoporosis.

In another hybridization method, Northern analysis (see Current Protocols in Molecular Biology, Ausubel, F. et al., eds., John Wiley & Sons, supra) is used to identify the presence of a polymorphism associated with a susceptibility to osteoporosis. For Northern analysis, a test sample of RNA is obtained from the individual by appropriate means. Specific hybridization of a nucleic acid probe, as described above, to RNA from the individual is indicative of a polymorphism in BMP2, and is therefore diagnostic for a susceptibility to osteoporosis.

For representative examples of use of nucleic acid probes, see, for example, U.S. Pat. Nos. 5,288,611 and 4,851,330.

Alternatively, a peptide nucleic acid (PNA) probe can be used instead of a nucleic acid probe in the hybridization methods described above. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen, P. E. et al., Bioconjugate Chemistry, 1994, 5, American Chemical Society, p. 1 (1994). The PNA probe can be designed to specifically hybridize to a gene having a polymorphism associated with a susceptibility to osteoporosis. Hybridization of the PNA probe to BMP2 is diagnostic for a susceptibility to osteoporosis.

In one embodiment of the invention, diagnosis of osteoporosis or a susceptibility to osteoporosis associated with BMP2, can be made by expression analysis using quantitative PCR (kinetic thermal cycling). In one embodiment, the diagnosis of osteoporosis is made by detecting at least one BMP2-associated allele and in combination with a bone turnover marker assay (e.g., bone scans). This technique can, for example, utilize commercially available technologies such as TaqMan® (Applied Biosystems, Foster City, Calif.), to allow the identification of, for example, polymorphisms. The technique can assess the presence of an alteration in the expression or composition of the polypeptide encoded by BMP2 or splicing variants. Further, the expression of the variants can be quantified as physically or functionally different.

In another method of the invention, mutation analysis by restriction digestion can be used to detect a mutant gene, or genes containing a polymorphism(s), if the mutation or polymorphism in the gene results in the creation or elimination of a restriction site. A test sample containing genomic DNA is obtained from the individual. Polymerase chain reaction (PCR) can be used to amplify BMP2 (and, if necessary, the flanking sequences) in the test sample of genomic DNA from the test individual. RFLP analysis is conducted as described (see Current Protocols in Molecular Biology, supra). The digestion pattern of the relevant DNA fragment indicates the presence or absence of the mutation or polymorphism in BMP2, and therefore indicates the presence or absence of this susceptibility to osteoporosis.

Sequence analysis can also be used to detect specific polymorphisms in BMP2. A test sample of DNA or RNA is obtained from the test individual. PCR or other appropriate methods can be used to amplify the gene, and/or its flanking sequences, if desired. The sequence of BMP2, or a fragment of the gene, or cDNA, or fragment of the cDNA, or mRNA, or fragment of the mRNA, is determined, using standard methods. The sequence of the gene, gene fragment, cDNA, cDNA fragment, mRNA, or mRNA fragment is compared with the known nucleic acid sequence of the gene, cDNA (e.g., SEQ ID NO: 1 and comprising at least one polymorphism as shown in Tables 2, 3 and 4) or mRNA, as appropriate. The presence of a specific polymorphism in BMP2 indicates that the individual has a susceptibility to osteoporosis.

Allele-specific oligonucleotides can also be used to detect the presence of a polymorphism in BMP2, through the use of dot-blot hybridization of amplified oligonucleotides with allele-specific oligonucleotide (ASO) probes (see, for example, Saiki, R. et al., (1986), Nature (London) 324:163-166). An “allele-specific oligonucleotide” (also referred to herein as an “allele-specific oligonucleotide probe”) is an oligonucleotide of approximately 10-50 base pairs or approximately 15-30 base pairs, that specifically hybridizes to BMP2, and that contains a polymorphism associated with a susceptibility to osteoporosis. An allele-specific oligonucleotide probe that is specific for particular polymorphisms in BMP2 can be prepared, using standard methods (see Current Protocols in Molecular Biology, supra). To identify polymorphisms in the gene that are associated with a susceptibility to osteoporosis, a test sample of DNA is obtained from the individual. PCR can be used to amplify all or a fragment of BMP2, and its flanking sequences. The DNA containing the amplified BMP2 (or fragment of the gene) is dot-blotted, using standard methods (see Current Protocols in Molecular Biology, supra), and the blot is contacted with the oligonucleotide probe. The presence of specific hybridization of the probe to the amplified BMP2 is then detected. Specific hybridization of an allele-specific oligonucleotide probe to DNA from the individual is indicative of a specific polymorphism in BMP2, and is therefore indicative of a susceptibility to osteoporosis.

An allele-specific primer hybridizes to a site on target DNA overlapping a polymorphism and only primes amplification of an allelic form to which the primer exhibits perfect complementarity (Gibbs, R et al., 1989. Nucleic Acids Res., 17:2437-2448). This primer is used in conjunction with a second primer, which hybridizes at a distal site. Amplification proceeds from the two primers, resulting in a detectable product, which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

With the addition of such analogs as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures of 64° C. and 74° C. when in complex with complementary DNA or RNA, respectively, as oposed to 28° C. for both DNA and RNA for the corresponding DNA nonamer. Substantial increases in Tₘ are also obtained when LNA monomers are used in combination with standard DNA or RNA monomers. For primers and probes, depending on where the LNA monomers are included (e.g., the 3′ end, the 5′ end, or in the middle), the Tₘ could be increased considerably.

In another embodiment, arrays of oligonucleotide probes that are complementary to target nucleic acid sequence segments from an individual, can be used to identify polymorphisms in a BMP2 nucleic acid. For example, in one embodiment, an oligonucleotide array can be used. Oligonucleotide arrays typically comprise a plurality of different oligonucleotide probes that are coupled to a surface of a substrate in different known locations. These oligonucleotide arrays, also described as “Genechips™,” have been generally described in the art, for example, U.S. Pat. No. 5,143,8.54 and PCT patent publication Nos. WO 90/15070 and 92/10092. These arrays can generally be produced using mechanical synthesis methods or light directed synthesis methods that incorporate a combination of photolithographic methods and solid phase oligonucleotide synthesis methods (Fodor, S. et al., 1991. Science, 251:767-773; Pirrung et al., U.S. Pat. No. 5,143,854 (see also PCT Application No. WO 90/15070); and Fodor. S. et al, PCT Publication No. WO 92/10092 and U.S. Pat. No. 5,424,186, the entire teachings of each of which are incorporated by reference herein). Techniques for the synthesis of these arrays using mechanical synthesis methods are described in, e.g., U.S. Pat. No. 5,384,261; the entire teachings of which are incorporated by reference herein. In another example, linear arrays can be utilized.

Once an oligonucleotide array is prepared, a nucleic acid of interest is hybridized with the array and scanned for polymorphisms. Hybridization and scanning are generally carried out by methods described herein and also in, e.g., published PCT Application Nos. WO 92/10092 and WO 95/11995, and U.S. Pat. No. 5,424,186, the entire teachings of which are incorporated by reference herein. In brief, a target nucleic acid sequence, which includes one or more previously identified polymorphic markers, is amplified by well known amplification techniques, e.g., PCR. Typically this involves the use of primer sequences that are complementary to the two strands of the target sequence, both upstream and downstream, from the polymorphism. Asymmetric PCR techniques can also be used. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Although primarily described in terms of a single detection block, e.g., for detection of a single polymorphism, arrays can include multiple detection blocks, and thus be capable of analyzing multiple, specific polymorphisms. In alternate arrangements, it will generally be understood that detection blocks can be grouped within a single array or in multiple, separate arrays so that varying, optimal conditions can be used during the hybridization of the target to the array. For example, it will often be desirable to provide for the detection of those polymorphisms that fall within G-C rich stretches of a genomic sequence, separately from those falling in A-T rich segments. This allows for the separate optimization of hybridization conditions for each situation.

Additional descriptions of use of oligonucleotide arrays for detection of polymorphisms can be found, for example, in U.S. Pat. Nos. 5,858,659 and 5,837,832, the entire teachings of which are incorporated by reference herein.

Other methods of nucleic acid analysis can be used to detect polymorphisms in BMP2. Representative methods include, for example, direct manual sequencing (Church and Gilbert, (1988), Proc. Natl. Acad. Sci. USA 81:1991-1995; Sanger, F. et al. (1977) Proc. Natl. Acad. Sci. 74:5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield, V. C. et al. (19891) Proc. Natl. Acad. Sci. USA 86:232-236), mobility shift analysis (Orita, M. et al (1989) Proc. Natl. Acad. Sci. USA 86:2766-2770), restriction enzyme analysis (Flavell et al. (1978) Cell 15:25; Geever, et al. (1981) Proc. Natl. Acad. Sci. USA 78:5081); heteroduplex analysis; chemical mismatch cleavage (CMC) (Cotton et al. (1985) Proc. Natl. Acad. Sci. USA 85:4397-4401); RNase protection assays (Myers, R. M. et al. (1985) Science 230:1242); use of polypeptides that recognize nucleotide mismatches, such as E. coli mutS protein; and allele-specific PCR.

In one embodiment of the invention, diagnosis of a disease or condition associated with a BMP2 nucleic acid (e.g., osteoporosis) or a susceptibility to a disease or condition associated with a BMP2 nucleic acid (e.g., osteoporosis) can also be made by expression analysis by quantitative PCR (kinetic thermal cycling). In one embodiment, the diagnosis of the disease itself, e.g., osteoporosis, is made by detecting at least one BMP2-associated allele and in combination with a bone turnover marker assay (e.g., bone scans). This technique, utilizing TaqMan®, can be used to allow the identification of polymorphisms and whether a patient is homozygous or heterozygous. The technique can assess the presence of an alteration in the expression or composition of the polypeptide encoded by a BMP2 nucleic acid or splicing variants encoded by a BMP2 nucleic acid. Further, the expression of the variants can be quantified as physically or functionally different.

In another embodiment of the invention, diagnosis of a susceptibility to osteoporosis can also be made by examining expression and/or composition of an BMP2 polypeptide, by a variety of methods, including enzyme linked immunosorbent assays (ELISAs), Western blots, immunoprecipitations and immunofluorescence. A test sample from an individual is assessed for the presence of an alteration in the expression and/or an alteration in composition of the polypeptide encoded by BMP2. An alteration in expression of a polypeptide encoded by BMP2 can be, for example, an alteration in the quantitative polypeptide expression (i.e., the amount of polypeptide produced); an alteration in the composition of a polypeptide encoded by BMP2 is an alteration in the qualitative polypeptide expression (e.g., expression of a mutant BMP2 polypeptide or of a different splicing variant). In one embodiment, diagnosis of a susceptibility to osteoporosis is made by detecting a particular splicing variant encoded by BMP2, or a particular pattern of splicing variants.

Both such alterations (quantitative and qualitative) can also be present. An “alteration” in the polypeptide expression or composition, as used herein, refers to an alteration in expression or composition in a test sample, as compared with the expression or composition of polypeptide by BMP2 in a control sample. A control sample is a sample that corresponds to the test sample (e.g., is from the same type of cells), and is from an individual who is not affected by osteoporosis. Similarly, the presence of one or more different splicing variants in the test sample, or the presence of significantly different amounts of different splicing variants in the test sample, as compared with the control sample, is indicative of a susceptibility to osteoporosis. An alteration in the expression or composition of the polypeptide in the test sample, as compared with the control sample, is indicative of a susceptibility to osteoporosis. Various means of examining expression or composition of the polypeptide encoded by BMP2 can be used, including spectroscopy, colorimetry, electrophoresis, isoelectric focusing, and immunoassays (e.g., David et al., U.S. Pat. No. 4,376,110) such as immunoblotting (see also Current Protocols in Molecular Biology, particularly chapter 10).

For example, in one embodiment, an antibody capable of binding to the polypeptide (e.g., as described above), e.g., an antibody with a detectable label, can be used. Antibodies can be polyclonal or monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Western blot analysis, using an antibody as described above that specifically binds to a polypeptide encoded by a mutant BMP2, or an antibody that specifically binds to a polypeptide encoded by a non-mutant gene, can be used to identify the presence in a test sample of a polypeptide encoded by a polymorphic or mutant BMP2, or the absence in a test sample of a polypeptide encoded by a non-polymorphic or non-mutant gene. The presence of a polypeptide encoded by a polymorphic or mutant gene, or the absence of a polypeptide encoded by a non-polymorphic or non-mutant gene, is diagnostic for a susceptibility to osteoporosis.

In one embodiment of this method, the level or amount of polypeptide encoded by BMP2 in a test sample is compared with the level or amount of the polypeptide encoded by BMP2 in a control sample. A level or amount of the polypeptide in the test sample that is higher or lower than the level or amount of the polypeptide in the control sample, such that the difference is statistically significant, is indicative of an alteration in the expression of the polypeptide encoded by BMP2, and is diagnostic for a susceptibility to osteoporosis. Alternatively, the composition of the polypeptide encoded by BMP2 in a test sample is compared with the composition of the polypeptide encoded by BMP2 in a control sample. A difference in the composition of the polypeptide in the test sample, as compared with the composition of the polypeptide in the control sample, is diagnostic for a susceptibility to osteoporosis. In another embodiment, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a susceptibility to osteoporosis.

Kits useful in the methods of diagnosis comprise components useful in any of the methods described herein, including for example, hybridization probes, restriction enzymes (e.g., for RFLP analysis), allele-specific oligonucleotides, antibodies which bind to altered or to non-altered (native) BMP2 polypeptide (e.g., to SEQ ID NO: 2 and comprising at least one polymorphism as shown in Tables 2, 3 and 4), means for amplification of nucleic acids comprising BMP2, or means for analyzing the nucleic acid sequence of BMP2 or for analyzing the amino acid sequence of an BMP2 polypeptide, etc. Additionally, kits can provide reagents for assays to be used in combination with the methods of the present invention, e.g., bone turnover marker assays (e.g., bone scans).

Kits (e.g., reagent kits) useful in the methods of diagnosis comprise components useful in any of the methods described herein, including for example, hybridization probes or primers as described herein (e.g., labeled probes or primers), reagents for detection of labeled molecules, restriction enzymes (e.g., for RFLP analysis), allele-specific oligonucleotides, antibodies that bind to altered or to non-altered (native) BMP2 polypeptide, means for amplification of nucleic acids comprising a BMP2, or means for analyzing the nucleic acid sequence of a BMP2 nucleic acid or for analyzing the amino acid sequence of a BMP2 polypeptide as described herein, etc. In one embodiment, the kit for diagnosing osteoporosis or a susceptibility to osteoporosis can comprise primers for nucleic acid amplification of a region in the BMP2 nucleic acid comprising one or more polymorphic sites that are more frequently present in an individual having osteoporosis or is susceptible to osteoporosis. The primers can be designed using portions of the nucleic acids flanking SNPs that are indicative of osteoporosis. Additionally, kits can provide reagents for assays to be used in combination with the methods of the present invention, e.g., bone turnover marker assays (e.g., bone scans).

The invention further pertains to a method for the diagnosis and identification of susceptibility to osteoporosis in an individual, by identifying an at-risk marker, e.g., an SNP, in BMP2. In one embodiment, the at-risk marker is one that confers a significant risk of osteoporosis. In one embodiment, significance associated with a marker is measured by an odds ratio. In a further embodiment, the significance is measured by a percentage. In one embodiment, a significant risk is measured as an odds ratio of at least about 2.2, including by not limited to: 1.2, 1.3, 1.4, 1.5, 1.6, 1.7, 1.8, and 1.9. In a further embodiment, an odds ratio of at least 1.2 is significant. In a further embodiment, an odds ratio of at least about 1.5 is significant. In a further embodiment, a significant increase in risk is at least about 1.7 is significant. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%. It is understood however, that identifying whether a risk is medically significant can also depend on a variety of factors, including the specific disease, the marker, and often, environmental factors.

The invention also pertains to methods of diagnosing osteoporosis or a susceptibility to osteoporosis in an individual, comprising screening for an at-risk marker associated with the BMP2 nucleic acid that is more frequently present in an individual susceptible to osteoporosis (affected), compared to the frequency of its presence in a healthy individual (control), wherein the presence of the marker is indicative of osteoporosis or susceptibility to osteoporosis. Standard techniques for genotyping for the presence of SNPs and/or microsatellite markers that are associated with osteoporosis can be used, such as fluorescent based techniques (Chen, et al., 1999. Genome Res., 9:492), PCR, LCR, Nested PCR and other techniques for nucleic acid amplification. In one embodiment, the method comprises assessing in an individual the presence or frequency of a specific SNP allele or microsatellite allele associated with the BMP2 nucleic acid that are associated with osteoporosis, wherein an excess or higher frequency of the marker compared to a healthy control individual is indicative that the individual has osteoporosis or is susceptible to osteoporosis. See Tables 2, 3 and 4 for SNPs and markers that can be used as screening tools.

Screening Assays and Agents Identified Thereby

The invention provides methods (also referred to herein as “screening assays”) for identifying the presence of a nucleotide that hybridizes to a nucleic acid of the invention, as well as for identifying the presence of a polypeptide encoded by a nucleic acid of the invention. In one embodiment, the presence (or absence) of a nucleic acid molecule of interest (e.g., a nucleic acid that has significant homology with a nucleic acid of the invention) in a sample can be assessed by contacting the sample with a nucleic acid comprising a nucleic acid of the invention (e.g., a nucleic acid having the sequence of SEQ ID NO: 1 and comprising at least one polymorphism as shown in Tables 2, 3 and 4, or the complement thereof, or a nucleic acid encoding an amino acid having the sequence of SEQ ID NO: 2 or a fragment or variant of such nucleic acids), under stringent conditions as described above, and then assessing the sample for the presence (or absence) of hybridization. In one embodiment, high stringency conditions are conditions appropriate for selective hybridization. In another embodiment, a sample containing the nucleic acid molecule of interest is contacted with a nucleic acid containing a contiguous nucleotide sequence (e.g., a primer or a probe as described above) that is at least partially complementary to a part of the nucleic acid molecule of interest (e.g., a variant BMP2 nucleic acid), and the contacted sample is assessed for the presence or absence of hybridization. In one embodiment, the nucleic acid containing a contiguous nucleotide sequence is completely complementary to a part of the nucleic acid molecule of interest.

In any of these embodiments, all or a portion of the nucleic acid of interest can be subjected to amplification prior to performing the hybridization.

In another embodiment, the presence (or absence) of a polypeptide of interest, such as a polypeptide of the invention or a fragment or variant thereof, in a sample can be assessed by contacting the sample with an antibody that specifically hybridizes to the polypeptide of interest (e.g., an antibody such as those described above), and then assessing the sample for the presence (or absence) of binding of the antibody to the polypeptide of interest.

In another embodiment, the invention provides methods for identifying agents (e.g., fusion proteins, polypeptides, peptidomimetics, prodrugs, receptors, binding agents, antibodies, small molecules or other drugs, or ribozymes) that alter (e.g., increase or decrease) the activity of the polypeptides described herein, or that otherwise interact with the polypeptides herein. For example, such agents can be agents that bind to polypeptides described herein (e.g., BMP2 binding agents); that have a stimulatory or inhibitory effect on, for example, activity of polypeptides of the invention; that change (e.g., enhance or inhibit) the ability of the polypeptides of the invention to interact with BMP2 binding agents (e.g., receptors or other binding agents); or that alter posttranslational processing of the BMP2 polypeptide (e.g., agents that alter proteolytic processing to direct the polypeptide from where it is normally synthesized to another location in the cell, such as the cell surface; agents that alter proteolytic processing such that more polypeptide is released from the cell, etc).

In one embodiment, the invention provides assays for screening candidate or test agents that bind to or modulate the activity of polypeptides described herein (or biologically active portion(s) thereof), as well as agents identifiable by the assays. Test agents can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des., 12: 145).

In one embodiment, a cell, cell lysate, or solution containing or expressing an BMP2 polypeptide (e.g., SEQ ID NO: 2 and comprising at least one polymorphism as shown in Tables 2, 3 or 4), or an active fragment or derivative thereof (as described above), can be contacted with an agent to be tested to identify agents which alter the activity of an BMP2 polypeptide; alternatively, the polypeptide can be contacted directly with the agent to be tested. The level (amount) of BMP2 activity is assessed (e.g., the level (amount) of BMP2 activity is measured, either directly or indirectly), and is compared with the level of activity in a control (i.e., the level of activity of the BMP2 polypeptide or active fragment or derivative thereof in the absence of the agent to be tested). If the level of the activity in the presence of the agent differs by an amount that is statistically significant from the level of the activity in the absence of the agent, then the agent is an agent that alters the activity of BMP2 polypeptide. An increase in the level of BMP2 activity relative to a control, indicates that the agent is an agent that enhances (is an agonist of) BMP2 activity. Similarly, a decrease in the level of BMP2 activity relative to a control, indicates that the agent is an agent that inhibits (is an antagonist of) BMP2 activity. In another embodiment, the level of activity of an BMP2 polypeptide or derivative or fragment thereof in the presence of the agent to be tested, is compared with a control level that has previously been established. A level of the activity in the presence of the agent that differs from the control level by an amount that is statistically significant indicates that the agent alters BMP2 activity.

The present invention also relates to an assay for identifying agents that alter the expression of the BMP2 nucleic acid (e.g., antisense nucleic acids, fusion proteins, polypeptides, peptidomimetics, prodrugs, receptors, binding agents, antibodies, small molecules or other drugs, or ribozymes), that alter (e.g., increase or decrease) expression (e.g., transcription or translation) of the gene, or that otherwise interact with the nucleic acids described herein, as well as agents identifiable by the assays. For example, a solution containing a nucleic acid encoding BMP2 polypeptide (e.g., BMP2 gene) can be contacted with an agent to be tested. The solution can comprise, for example, cells containing the nucleic acid or cell lysate containing the nucleic acid; alternatively, the solution can be another solution that comprises elements necessary for transcription/translation of the nucleic acid. Cells not suspended in solution can also be employed, if desired. The level and/or pattern of BMP2 expression (e.g., the level and/or pattern of mRNA or of protein expressed, such as the level and/or pattern of different splicing variants) is assessed, and is compared with the level and/or pattern of expression in a control (i.e., the level and/or pattern of the BMP2 expression in the absence of the agent to be tested). If the level and/or pattern in the presence of the agent differs, by an amount or in a manner that is statistically significant, from the level and/or pattern in the absence of the agent, then the agent is an agent that alters the expression of BMP2. Enhancement of BMP2 expression indicates that the agent is an agonist of BMP2 activity. Similarly, inhibition of BMP2 expression indicates that the agent is an antagonist of BMP2 activity. In another embodiment, the level and/or pattern of BMP2 polypeptide(s)(e.g., different splicing variants) in the presence of the agent to be tested, is compared with a control level and/or pattern that has previously been established. A level and/or pattern in the presence of the agent that differs from the control level and/or pattern by an amount or in a manner that is statistically significant indicates that the agent alters BMP2 expression.

In another embodiment of the invention, agents that alter the expression of the BMP2 nucleic acid or that otherwise interact with the nucleic acids described herein, can be identified using a cell, cell lysate, or solution containing a nucleic acid encoding the promoter region of the BMP2 nucleic acid operably linked to a reporter gene. After contact with an agent to be tested, the level of expression of the reporter gene (e.g., the level of mRNA or of protein expressed) is assessed, and is compared with the level of expression in a control (i.e., the level of the expression of the reporter gene in the absence of the agent to be tested). If the level in the presence of the agent differs, by an amount or in a manner that is statistically significant, from the level in the absence of the agent, then the agent is an agent that alters the expression of BMP2, as indicated by its ability to alter expression of a gene that is operably linked to the BMP2 gene promoter. Enhancement of the expression of the reporter indicates that the agent is an agonist of BMP2 activity. Similarly, inhibition of the expression of the reporter indicates that the agent is an antagonist of BMP2 activity. In another embodiment, the level of expression of the reporter in the presence of the agent to be tested, is compared with a control level that has previously been established. A level in the presence of the agent that differs from the control level by an amount or in a manner that is statistically significant indicates that the agent alters BMP2 expression.

Agents that alter the amounts of different splicing variants encoded by BMP2 (e.g., an agent that enhances the activity of a first splicing variant, and that inhibits activity of a second splicing variant), as well as agents that are agonists of activity of a first splicing variant and antagonists of activity of a second splicing variant, can be identified using these methods described above.

In other embodiments of the invention, assays can be used to assess the impact of a test agent on the activity of a polypeptide in relation to a BMP2 binding agent. For example, a cell that expresses a compound that interacts with BMP2 (herein referred to as a “BMP2 binding agent”, which can be a polypeptide or other molecule that interacts with BMP2, such as a receptor) is contacted with BMP2 in the presence of a test agent, and the ability of the test agent to alter the interaction between BMP2 and the BMP2 binding agent is determined. Alternatively, a cell lysate or a solution containing the BMP2 binding agent, can be used. An agent that binds to BMP2 or the BMP2 binding agent can alter the interaction by interfering with, or enhancing the ability of BMP2 to bind to, associate with, or otherwise interact with the BMP2 binding agent. Determining the ability of the test agent to bind to BMP2 or an BMP2 binding agent can be accomplished, for example, by coupling the test agent with a radioisotope or enzymatic label such that binding of the test agent to the polypeptide can be determined by detecting the labeled with ¹²⁵I, ³⁵S, ³²P, ³³P, ¹⁴C or ³H, either directly or indirectly, and the radioisotope detected by direct counting of radioemmission or by scintillation counting. Alternatively, test agents can be enzymatically labeled with, for example, horseradish peroxidase, alkaline phosphatase, or luciferase, and the enzymatic label detected by determination of conversion of an appropriate substrate to product. It is also within the scope of this invention to determine the ability of a test agent to interact with the polypeptide without the labeling of any of the interactants. For example, a microphysiometer can be used to detect the interaction of a test agent with BMP2 or a BMP2 binding agent without the labeling of either the test agent, BMP2, or the BMP2 binding agent (McConnell, H. M. et al (1992) Science, 257:1906-1912). As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between ligand and polypeptide. See the “Examples” section for a discussion of know BMP2 binding-partners. Thus, these receptors can be used to screen for compounds that are BMP2 receptor agonists for use in treating osteoporosis or BMP2 receptor antagonists for studying osteoporosis. The linkage data provided herein, for the first time, provides such correction to osteoporosis. Drugs could be designed to regulate BMP2 receptor activation, and, in turn, could be used to regulate signaling pathways and transcription events of genes downstream.

In another embodiment of the invention, assays can be used to identify polypeptides that interact with one or more BMP2 polypeptides, as described herein. For example, a yeast two-hybrid system (Fields, S. and Song, O., Nature 340:245-246 (1989)) can be used to identify polypeptides that interact with one or more BMP2 polypeptides. In such a yeast two-hybrid system, vectors are constructed based on the flexibility of a transcription factor that has two functional domains (a DNA binding domain and a transcription activation domain). If the two domains are separated but fused to two different proteins that interact with one another, transcriptional activation can be achieved, and transcription of specific markers (e.g., nutritional markers such as His and Ade, or color markers such as lacZ) can be used to identify the presence of interaction and transcriptional activation. For example, in the methods of the invention, a first vector is used that includes a nucleic acid encoding a DNA binding domain and also an BMP2 polypeptide, splicing variant, or fragment or derivative thereof, and a second vector is used that includes a nucleic acid encoding a transcription activation domain and also a nucleic acid encoding a polypeptide that potentially can interact with the BMP2 polypeptide, splicing variant, or fragment or derivative thereof (e.g., a BMP2 polypeptide binding agent or receptor). Incubation of yeast containing the first vector and the second vector under appropriate conditions (e.g., mating conditions such as used in the Matchmaker™ system from Clontech) allows identification of colonies that express the markers of interest. These colonies can be examined to identify the polypeptide(s) that interact with the BMP2 polypeptide or fragment or derivative thereof. Such polypeptides can be useful as agents that alter the activity of expression of an BMP2 polypeptide, as described above.

In more than one embodiment of the above assay methods of the present invention, it could be desirable to immobilize either BMP2, the BMP2 binding agent, or other components of the assay on a solid support in order to facilitate separation of complexed from uncomplexed forms of one or both of the polypeptides, as well as to accommodate automation of the assay. Binding of a test agent to the polypeptide, or interaction of the polypeptide with a binding agent in the presence and absence of a test agent, can be accomplished in any vessel suitable for containing the reactants. Examples of such vessels include microtitre plates, test tubes, and micro-centrifuge tubes. In one embodiment, a fusion protein (e.g., a GST fusion protein) can be provided, thus adding a domain that allows BMP2 or a BMP2 binding agent to be bound to a matrix or other solid support.

In another embodiment, modulators of expression of nucleic acid molecules of the invention are identified in a method wherein a cell, cell lysate, or solution containing a nucleic acid encoding BMP2 is contacted with a test agent and the expression of appropriate mRNA or polypeptide (e.g., splicing variant(s)) in the cell, cell lysate, or solution, is determined. The level of expression of appropriate mRNA or polypeptide(s) in the presence of the test agent is compared to the level of expression of mRNA or polypeptide(s) in the absence of the test agent. The test agent can then be identified as a modulator of expression based on this comparison. For example, when expression of mRNA or polypeptide is greater (statistically significantly greater) in the presence of the test agent than in its absence, the test agent is identified as a stimulator or enhancer of the mRNA or polypeptide expression. Alternatively, when expression of the mRNA or polypeptide is less (statistically significantly less) in the presence of the test agent than in its absence, the test agent is identified as an inhibitor of the mRNA or polypeptide expression. The level of mRNA or polypeptide expression in the cells can be determined by methods described herein for detecting mRNA or polypeptide.

This invention further pertains to novel agents identified by the above-described screening assays. Accordingly, it is within the scope of this invention to further use an agent identified as described herein in an appropriate animal model. For example, an agent identified as described herein (e.g., a test agent that is a modulating agent, an antisense nucleic acid molecule, a specific antibody, or a polypeptide-binding agent) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein. In addition, an agent identified as described herein can be used to alter activity of a polypeptide encoded by BMP2, or to alter expression of BMP2, by contacting the polypeptide or the gene (or contacting a cell comprising the polypeptide or the gene) with the agent identified as described herein.

Pharmaceutical Compositions

The present invention also pertains to pharmaceutical compositions comprising nucleic acids described herein, particularly nucleotides encoding the polypeptides described herein; comprising polypeptides described herein (e.g., SEQ ID NO: 2); and/or comprising the agent that alters (e.g., enhances or inhibits) BMP2 polypeptide activity described herein. For instance, a polypeptide, protein, fragment, fusion protein or prodrug thereof, or a nucleotide or nucleic acid construct (vector) comprising a nucleotide of the present invention, or an agent that alters BMP2 polypeptide activity, can be formulated with a physiologically acceptable carrier or excipient to prepare a pharmaceutical composition. The carrier and composition can be sterile. The formulation should suit the mode of administration.

Suitable pharmaceutically acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active agents.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrolidone, sodium saccharine, cellulose, magnesium carbonate, etc.

Methods of introduction of these compositions include, but are not limited to, intradermal, intramuscular, intraperitoneal, intraocular, intravenous, subcutaneous, topical, oral and intranasal. Other suitable methods of introduction can also include gene therapy (as described below), rechargeable or biodegradable devices, particle acceleration devises (“gene guns”) and slow release polymeric devices. The pharmaceutical compositions of this invention can also be administered as part of a combinatorial therapy with other agents.

The composition can be formulated in accordance with the routine procedures as a pharmaceutical composition adapted for administration to human beings. For example, compositions for intravenous administration typically are solutions in sterile isotonic aqueous buffer. Where necessary, the composition can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients can be mixed prior to administration.

For topical application, nonsprayable forms, viscous to semi-solid or solid forms comprising a carrier compatible with topical application and having a dynamic viscosity greater than water, can be employed. Suitable formulations include but are not limited to solutions, suspensions, emulsions, creams, ointments, powders, enemas, lotions, sols, liniments, salves, aerosols, etc., which are, if desired, sterilized or mixed with auxiliary agents, e.g., preservatives, stabilizers, wetting agents, buffers or salts for influencing osmotic pressure, etc. The agent can be incorporated into a cosmetic formulation. For topical application, also suitable are sprayable aerosol preparations wherein the active ingredient, optionally in combination with a solid or liquid inert carrier material, is packaged in a squeeze bottle or in admixture with a pressurized volatile, normally gaseous propellant, e.g., pressurized air.

Agents described herein can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The agents are administered in a therapeutically effective amount. The amount of the agent that will be therapeutically effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the formulation will also depend on the route of administration, and the seriousness of the symptoms of osteoporosis, and should be decided according to the judgment of a practitioner and each patient's circumstances. Effective doses can be extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use of sale for human administration. The pack or kit can be labeled with information regarding mode of administration, sequence of drug administration (e.g., separately, sequentially or concurrently), or the like. The pack or kit can also include means for reminding the patient to take the therapy. The pack or kit can be a single unit dosage of the combination therapy or it can be a plurality of unit dosages. In particular, the agents can be separated, mixed together in any combination, present in a single vial or tablet. Agents assembled in a blister pack or other dispensing means is envisioned by the present invention. For the purpose of this invention, unit dosage is intended to mean a dosage that is dependent on the individual pharmacodynamics of each agent and administered in FDA approved dosages in standard time courses.

Methods of Therapy

The present invention also pertains to methods of treatment (prophylactic and/or therapeutic) for osteoporosis or a susceptibility to osteoporosis, using an BMP2 therapeutic agent. A “BMP2 therapeutic agent” is an agent that alters (e.g., enhances or inhibits) BMP2 polypeptide activity and/or BMP2 expression, as described herein (e.g., an BMP2 agonist or antagonist).

BMP2 therapeutic agents can alter BMP2 polypeptide activity or gene expression by a variety of means, such as, for example, by providing additional BMP2 polypeptide or by upregulating the transcription or translation of the BMP2 gene; by altering posttranslational processing of the BMP2 polypeptide; by altering transcription of BMP2 splicing variants; or by interfering with BMP2 polypeptide activity (e.g., by binding to a BMP2 polypeptide), or by downregulating the transcription or translation of the BMP2 gene. Representative BMP2 therapeutic agents include the following: nucleic acids or fragments or derivatives thereof described herein, particularly nucleotides encoding the polypeptides described herein and vectors comprising such nucleic acids (e.g., a gene, cDNA, and/or mRNA, such as a nucleic acid encoding a BMP2 polypeptide or active fragment or derivative thereof, or an oligonucleotide; for example, SEQ ID NO: 1 and optionally comprising at least one polymorphism as shown in Tables 2, 3 and 4 or a nucleic acid encoding SEQ ID NO: 2 and optionally comprising at least one polymorphism as shown in Tables 2, 3 and 4, or fragments or derivatives thereof); polypeptides described herein (e.g., SEQ ID NO: 2 comprising at least one polymorphism as shown in Tables 2, 3 and 4, and/or other splicing variants encoded by BMP2, or fragments or derivatives thereof); other polypeptides (e.g., BMP2 receptors); BMP2 binding agents, including but not limited to BMP2R2, BMPR1A and BMPR1B; peptidomimetics; fusion proteins or prodrugs thereof, antibodies (e.g., an antibody to a mutant BMP2 polypeptide, or an antibody to a non-mutant BMP2 polypeptide, or an antibody to a particular splicing variant encoded by BMP2, as described above); ribozymes; other small molecules; and other agents that alter (e.g., enhance or inhibit) BMP2 gene expression or polypeptide activity, or that regulate transcription of BMP2 splicing variants (e.g., agents that affect which splicing variants are expressed, or that affect the amount of each splicing variant that is expressed.

The BMP2R2, BMPR1A and BMPR1B are cell-surface receptors binding the BMP2 protein; the BMP2 protein binds to these receptors, and, in turn, stimulates certain intracellular responses (Fujii, M., et al., Mol. Biol. Cell, 10(11):3801-13 (1999); Massague, J., Annu. Rev. Biochem., 67:753-91 (1998); Chen, D., et al., J. Cell Biol., 142(1):295-305 (1998); and Kirsch, T., et. al., Nature Structural Biology, 7(6):492-496 (2000)). There are also known direct inhibitors of BMP2, e.g., noggin and chordin (at least noggin; chordin is a known inhibitor of other BMPs) (Zimmerman, L. B., et. al., Cell, 86:599-606 (1996); Aspenberg, P., et. al., J. Bone Miner. Res., 16(3):497-500 (2001); and Dale., L. et. al., Bioessays, 21(9):751-60 (1999)).

More than one BMP2 therapeutic agent can be used concurrently with another, if desired.

The BMP2 therapeutic agent that is a nucleic acid is used in the treatment of osteoporosis or in the treatment for a susceptibility to osteoporosis. The term, “treatment” as used herein, refers not only to ameliorating symptoms associated with the disease, but also preventing or delaying the onset of the disease, and also lessening the severity or frequency of symptoms of the disease. The therapy is designed to alter (e.g., inhibit or enhance), replace or supplement activity of a BMP2 polypeptide in an individual. For example, a BMP2 therapeutic agent can be administered in order to upregulate or increase the expression or availability of the BMP2 gene or of specific splicing variants of BMP2, or, conversely, to downregulate or decrease the expression or availability of the BMP2 gene or specific splicing variants of BMP2. Upregulation or increasing expression or availability of a native BMP2 nucleic acid or of a particular splicing variant could interfere with or compensate for the expression or activity of a defective gene or another splicing variant; downregulation or decreasing expression or availability of a native BMP2 nucleic acid or of a particular splicing variant could minimize the expression or activity of a defective gene or the particular splicing variant and thereby minimize the impact of the defective gene or the particular splicing variant. In one embodiment, the BMP2 therapeutic agent is the healthy gene or gene product (SEQ ID NO: 2), e.g., a BMP2 nucleic acid and gene product that is not associated with osteoporosis.

The BMP2 therapeutic agent(s) are administered in a therapeutically effective amount (e.g., an amount that is sufficient to treat the disease, such as by ameliorating symptoms associated with the disease, preventing or delaying the onset of the disease, and/or also lessening the severity or frequency of symptoms of the disease). The amount that will be therapeutically effective in the treatment of a particular individual's disorder or condition will depend on the symptoms and severity of the disease, and can be determined by standard clinical techniques. In addition, in vitro or in vivo assays can optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses can be extrapolated from dose-response curves derived from in vitro or animal model test systems.

In one embodiment, a nucleic acid of the invention (e.g., a nucleic acid encoding a BMP2 polypeptide, such as SEQ ID NO: 1 optionally comprising at least one polymorphism as shown in Tables 2, 3 and 4 or another nucleic acid that encodes a BMP2 polypeptide or a splicing variant, derivative or fragment thereof, such as a nucleic acid encoding SEQ ID NO: 2 and comprising at least one polymorphism as shown in Tables 2, 3 and 4 can be used, either alone or in a pharmaceutical composition as described above. For example, BMP2 or a cDNA encoding the BMP2 polypeptide, either by itself or included within a vector, can be introduced into cells (either in vitro or in vivo) such that the cells produce native BMP2 polypeptide. If necessary, cells that have been transformed with the gene or cDNA or a vector comprising the gene or cDNA can be introduced (or re-introduced) into an individual affected with the disease. Thus, cells that, in nature, lack native BMP2 expression and activity, or have mutant BMP2 expression and activity, or have expression of a disease-associated BMP2 splicing variant, can be engineered to express BMP2 polypeptide or an active fragment of the BMP2 polypeptide (or a different variant of BMP2 polypeptide). In one embodiment, a nucleic acid encoding the BMP2 polypeptide, or an active fragment or derivative thereof, can be introduced into an expression vector, such as a viral vector, and the vector can be introduced into appropriate cells in an animal. Other gene transfer systems, including viral and nonviral transfer systems, can be used. Alternatively, nonviral gene transfer methods, such as calcium phosphate coprecipitation, mechanical techniques (e.g., microinjection); membrane fusion-mediated transfer via liposomes; or direct DNA uptake, can also be used.

Alternatively, in another embodiment of the invention, a nucleic acid of the invention; a nucleic acid complementary to a nucleic acid of the invention; or a portion of such a nucleic acid (e.g., an oligonucleotide as described below), can be used in “antisense” therapy, in which a nucleic acid (e.g., an oligonucleotide) that specifically hybridizes to the mRNA and/or genomic DNA of BMP2 is administered or generated in situ. The antisense nucleic acid that specifically hybridizes to the mRNA and/or DNA inhibits expression of the BMP2 polypeptide, e.g., by inhibiting translation and/or transcription. Binding of the antisense nucleic acid can be by conventional base pair complementarity, or, for example, in the case of binding to DNA duplexes, through specific interaction in the major groove of the double helix.

An antisense construct of the present invention can be delivered, for example, as an expression plasmid as described above. When the plasmid is transcribed in the cell, it produces RNA that is complementary to a portion of the mRNA and/or DNA that encodes BMP2 polypeptide. Alternatively, the antisense construct can be an oligonucleotide probe that is generated ex vivo and introduced into cells; it then inhibits expression by hybridizing with the mRNA and/or genomic DNA of BMP2. In one embodiment, the oligonucleotide probes are modified oligonucleotides that are resistant to endogenous nucleases, e.g. exonucleases and/or endonucleases, thereby rendering them stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy are also described, for example, by Van der Krol et al. ((1988) Biotechniques 6:958-976); and Stein et al ( (1988) Cancer Res 48:2659-2668). With respect to antisense DNA, oligodeoxyribonucleotides derived from the translation initiation site, e.g. between the −10 and +10 regions of BMP2 sequence, can be utilized.

To perform antisense therapy, oligonucleotides (mRNA, cDNA or DNA) are designed that are complementary to mRNA encoding BMP2. The antisense oligonucleotides bind to BMP2 mRNA transcripts and prevent translation. Absolute complementarity is not required. A sequence “complementary” to a portion of an RNA, as referred to herein, indicates that a sequence has sufficient complementarity to be able to hybridize with the RNA, forming a stable duplex; in the case of double-stranded antisense nucleic acids, a single strand of the duplex DNA can thus be tested, or triplex formation can be assayed. The ability to hybridize will depend on both the degree of complementarity and the length of the antisense nucleic acid, as described in detail above. Generally, the longer the hybridizing nucleic acid, the more base mismatches with an RNA it can contain and still form a stable duplex (or triplex, as the case may be). One skilled in the art can ascertain a tolerable degree of mismatch by use of standard procedures.

The oligonucleotides used in antisense therapy can be DNA, RNA, or chimeric mixtures or derivatives or modified versions thereof, single-stranded or double-stranded. The oligonucleotides can be modified at the base moiety, sugar moiety, or phosphate backbone, for example, to improve stability of the molecule, hybridization, etc. The oligonucleotides can include other appended groups such as peptides (e.g. for targeting host cell receptors in vivo), or agents facilitating transport across the cell membrane (see, e.g., Letsinger et al. (1989) Proc. Natl. Acad. Sci. USA 86:6553-6556; Lemaitre et al., (1987), Proc. Natl. Acad. Sci. USA 84:648-652; PCT International Publication No. W088/09810) or the blood-brain barrier (see, e.g., PCT International Publication No. W089/10134), or hybridization-triggered cleavage agents (see, e.g., Krol et al. (1988) BioTechniques 6:958-976) or intercalating agents. (See, e.g., Zon, (1988), Pharm. Res. 5:539-549). To this end, the oligonucleotide can be conjugated to another molecule (e.g., a peptide, hybridization triggered cross-linking agent, transport agent, hybridization-triggered cleavage agent).

The antisense molecules are delivered to cells that express BMP2 in vivo. A number of methods can be used for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically. Alternatively, in another embodiment, a recombinant DNA construct is utilized in which the antisense oligonucleotide is placed under the control of a strong promoter (e.g., pol III or pol II). The use of such a construct to transfect target cells in the patient results in the transcription of sufficient amounts of single-stranded RNAs that will form complementary base pairs with the endogenous BMP2 transcripts and thereby prevent translation of the BMP2 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art and described above. For example, a plasmid, cosmid, YAC or viral vector can be used to prepare the recombinant DNA construct that can be introduced directly into the tissue site. Alternatively, viral vectors can be used to selectively infect the desired tissue, in which case administration can be accomplished by another route (e.g., systemically).

Endogenous BMP2 expression can also be reduced by inactivating or “knocking out” BMP2 or its promoter using targeted homologous recombination (e.g., see Smithies et al. (1985) Nature 317:230-234; Thomas & Capecchi (1987) Cell 51:503-512; Thompson et al. (1989) Cell 5:313-321). For example, a mutant, non-functional BMP2 (or a completely unrelated DNA sequence) flanked by DNA homologous to the endogenous BMP2 (either the coding regions or regulatory regions of BMP2) can be used, with or without a selectable marker and/or a negative selectable marker, to transfect cells that express BMP2 in vivo. Insertion of the DNA construct, via targeted homologous recombination, results in inactivation of BMP2. The recombinant DNA constructs can be directly administered or targeted to the required site in vivo using appropriate vectors, as described above. Alternatively, expression of non-mutant BMP2 can be increased using a similar method: targeted homologous recombination can be used to insert a DNA construct comprising a non-mutant, functional BMP2 (e.g., a nucleic acid having SEQ ID NO: 1), or a portion thereof, in place of a mutant BMP2 in the cell, as described above. In another embodiment, targeted homologous recombination can be used to insert a DNA construct comprising a nucleic acid that encodes a BMP2 polypeptide variant that differs from that present in the cell.

Alternatively, endogenous BMP2 expression can be reduced by targeting deoxyribonucleotide sequences complementary to the regulatory region of BMP2 (i.e., the BMP2 promoter and/or enhancers) to form triple helical structures that prevent transcription of BMP2 in target cells in the body. (See generally, Helene, C. (1991) Anticancer Drug Des., 6(6):569-84; Helene, C., et al. (1992) Ann, N.Y. Acad. Sci., 660:27-36; and Maher, L. J. (1992) Bioassays 14(12):807-15). Likewise, the antisense constructs described herein, by antagonizing the normal biological activity of one of the BMP2 proteins, can be used in the manipulation of tissue, e.g. tissue differentiation, both in vivo and for ex vivo tissue cultures. Furthermore, the anti-sense techniques (e.g. microinjection of antisense molecules, or transfection with plasmids whose transcripts are anti-sense with regard to a BMP2 mRNA or gene sequence) can be used to investigate role of BMP2 in developmental events, as well as the normal cellular function of BMP2 in adult tissue. Such techniques can be utilized in cell culture, but can also be used in the creation of transgenic animals.

In yet another embodiment of the invention, other BMP2 therapeutic agents as described herein can also be used in the treatment or prevention of osteoporosis. The therapeutic agents can be delivered in a composition, as described above, or by themselves. They can be administered systemically, or can be targeted to a particular tissue. The therapeutic agents can be produced by a variety of means, including chemical synthesis; recombinant production; in vivo production (e.g., a transgenic animal, such as U.S. Pat. No. 4,873,316 to Meade et al.), for example, and can be isolated using standard means such as those described herein.

A combination of any of the above methods of treatment (e.g., administration of non-mutant BMP2 polypeptide in conjunction with antisense therapy targeting mutant BMP2 mRNA; administration of a variant encoded by BMP2 in conjunction with antisense therapy targeting a second variant encoded by BMP2), can also be used.

The invention will be further described by the following non-limiting examples. The teachings of all publications cited herein are incorporated herein by reference in their entirety.

### EXAMPLES

### Example 1

Identification of the BMP2 Nucleic Acid with Linkage to Osteoporosis

Phenotype and Family Construction

Patients who have low impact fractures and/or take bisphosphonates for treating osteoporosis are automatically treated as affecteds. People with low bone mass density (BMD) measurements are considered to be osteoporotic, and have been shown to have substantially increased risk of fractures. BMD measurements are taken for both the hip and the spine. For each person with BMD measurements, a standardized BMD score is computed (mean 0, standard deviation 1 for the population), which is adjusted for sex, age, body weight and hormone replacement therapy (HRT). For the combined analysis, the two measurements are summed. Population BMD data from Iceland and the United States are used for standardization and adjustment. For example, a person with a positive BMD score is above average and one with a negative score is below average for his/her age, body weight and possibly HRT. Assuming approximate normality, a score of −1 corresponds approximately to the lower 16^{th} percentile, etc.

For analysis, we start with a current list of primary people, people who have BMD measurements and/or are severely affected, and for whom we have genotypes. We then use the genealogy database to create family clusters linking these primary people using a threshold distance of 5 meiotic events. This procedure produced 190 potentially informative clusters with a total of 1215 primary people.

Linkage Data

Four genome wide scans (GWS) were performed using osteoporotic phenotypes at different skeletal sites; the hip, the spine, and combined phenotypes. All GWS analysis located at 20 cM region on Chr20, between 10 cM and 30 cM based on the Marshfield map.

All of the analyses were performed using the Allegro linkage program developed at deCODE (Gudbjartsson et al., Nature Genetics, 25: 12-13, May 2000). The allele sharing analysis uses the Sₚₐᵢᵣₛ scoring function of GENEHUNTER (Kruglyak et al., Am. J. Hum. Genet., 46: 1347-1363, 1996), but families were weighted using a scheme that is a compromise between weighting families equally and weighting affected pairs equally. The allele-sharing LOD scores were computed using the ‘exponential model’ described in Kong and Cox, Am. J. Hum. Genet., 61: 1179-1188 (1997).

Hip

The phenotype used was age, sex, weight and HRT corrected BMD<−1 SD at the hip (total hip). Hip fracture cases and bisphosphonate users are also considered affected even if values are above −1 SD. A total of 346 affected were used in this analysis. The GWS resulted in a LOD score of 3.1 using our standard set of markers. Adding 10 extra markers at the region on interest, between 11 cM and 39 cM, resulted in a LOD score of 3.3.

Spine

The phenotype was age, sex, weight and HRT corrected BMD<−1 SD at lumbar spine (L2-L4). Vertebral compression fracture cases and bisphosponate users are also considered affected even if values are above −1 SD. A total of 402 affected people were used in this analysis. The GWS resulted in a LOD score of 2.4 at the same location as in the hip analysis using the standard set of markers, but a LOD score of 2.9 with the extra marker set.

Combined

The phenotype used was the sum of corrected BMD<−1.5 SD. Vertebral compression fracture, hip fracture, other osteoporosis related low impact fracture (at least two fractures) and bisphosphonate users (BMD measurements before treatment start are used if available) are all considered affected. A total of 522 affected were used in this analysis. The GWS resulted in a LOD score of 2.5 with the standard marker set, but a LOD score of 3.9 using the extra markers in the region.

Combined Severe

The phenotype used was the sum of the age, sex, weight and HRT corrected BMD<−2.3 SD. Vertebral compression fracture, hip fracture, other osteoporosis related low impact fracture (at least two fractures) and bisphosphonate users affected. The number of affected in this analysis was 290. The GWS resulted in a LOD score of 3.8 with the standard set but a LOD score of 4.7 was reached using the extra 10 markers in addition.

Corticosteroid users and women with early menopause were excluded as affected in all analysis.

The BMP2 Gene

The BMP2 nucleic acid is located in this region. Only 5 kb are between the marker D20S846, which gives the highest LOD score, and the 3′ end of the gene. The gene has been sequenced and characterized in terms of exon/intron structures, promoter region and transcriptional start sites. This information are publicly available.

A number of nucleotide changes are observed in the Icelandic population. These changes have not to our knowledge been described before (See Table 2).

BMP2 binds to the receptors BMPR-IA or BMPR-IB, and BMPR-II, leading to formation of receptor complex heterodimer and phosphorylation of the BMPR-IA or BMPR-IB receptors. Once activated, these receptors subsequently phosphorylate SMAD1, SMAD5 or SMAD8, which in turn form complexes with SMAD4 and translocate to the nucleus where the transcription of specific genes is affected (Massague, J., Annu. Rev. Biochem., 67:753-791 (1998); Chen, D. et al., J. Cell Biol., 142(1):295-305 (1998)). SMADs 6 and 7 block signals by preventing the activation of SMAD1, SMAD5 or SMAD8 by the BMP2 receptors and have been shown to inhibit osteoblast differentiation (Miyazono, K., Bone, 25(1):91-93 (1999); Fujii, M., et al., Mol. Biol. Cell, 10(11):3801-3813 (1999)). BMP2 stimulates Cbfal, alkaline phosphatase and Collagen type I (osteoblast specific proteins) expression through BMPR-IB (Chen, D. et al., J. Cell Biol., 142(1):295-305 (1998). Cbfal regulates the expression of osteoprotegerin (OPG), which is an osteoblast-secreted glycoprotein that functions as a potent inhibitor of osteoblast differentiation and thus of bone resorption (Thirunavukkarasu, K., et al., J. Biol. Chem., (2000)). Cbfal controls osteoblast differentiation and bone formation. During cellular aging of human osteoblasts, there is a significant reduction (up to 50%) of Cbfal mRNA (Christiansen, M., et al., J. Gerontol. A Biol. Sci. Med. Sci., 55(4):B194-200 (2000)).

Results and Discussion

As a result of the linkage studies, the analysis shows that this locus is involved in multiple osteoporosis phenotypes. Furthermore, mutation within the human BMP2 nucleic acid is likely to explain the phenotypes in these families. Sporadic occurrence of osteoporosis, i.e., occurrence without familial connection, can also be determined using the information contained herein.

Osteoporosis could be caused by a defect in the BMP2 nucleic acid as follows: An alteration in the BMP2 nucleic acid (transcription, splice, protein variant etc.) could lead to a reduction of its action on Cbfal through BMPR-IB and the subsequent signaling pathway. This would lead to less bone formation because of fewer and less active osteoblasts and more bone resorption because of less OPG and more osteoclasts. This would lead to bone loss. Since a significant reduction of Cbfal levels is associated with aging osteoblasts, this effect could become more important with older age.

TABLE 1

| LOCUS | 14759 bp DNA |
|---|---|
| DEFINITION | Human bone morphogenetic protein 2 (BMP2) gene, |
| | complete cds, |
| | complete sequence. |
| ACCESSION | |
| VERSION | |
| KEYWORDS | . |
| SOURCE | human. |
| ORGANISM | Homo sapiens |
| | Eukaryota; Metazoa; Chordata; Craniata; Vertebrata; |
| | Mammalia; Eutheria; Primates; Catarrhini; Hominidae; |
| | Homo. |
| REFERENCE | 1 (bases 1-14759) |
| AUTHORS | Blakey, S. |
| TITLE | Direct Submission |
| JOURNAL | Submitted (04-APR-2000) Sanger Centre, Hinxton, |
| | Cambridgeshire, CB10 1SA, UK. E-mail enquiries: |
| | humquery@sanger.ac.uk Clone requests: |
| | clonerequest@sanger.ac.uk |
| COMMENT | This sequence was taken from GenBank sequence |
| | AL035668 (VERSION AL035668.15, GI:4995292), bp |
| | 118501..133259. |

| FEATURES | | Location/Qualifiers |
|---|---|---|
| | source | 1.. 14759 |
| | | /organism=“Homo sapiens” |
| | | /db_xref=“taxon : 9606” |
| | | /chromosome=“20” |
| | | /map=“20p12” |
| | | /clone=“RP5-859D4” |
| | | /clone_lib=“RPCI-5” |
| | gene | 2072..12634 |
| | | /gene=“BMP2” |
| | | /note=“BMP2A” |
| | | /db_xref=“LocusID:650” |
| | | /db_xref=“MIM:112261” |
| | exon | 2072..2387 |
| | | /gene=“BMP2” |
| | | /number=1 |
| | exon | 3632..3984 |
| | | /gene=“BMP2” |
| | | /number=2 |
| | CDS | /join(3639..3984, 11757..12601) |
| | | /gene=“BMP2” |
| | | /note=“BMP2 exons defined by |
| | | comparison to mRNA |

sequence (NM_001200)”

/codon_start=1
/product=“bone morphogenetic protein 2
precursor”
/protein_id=“NP_001191.1”
/db_xref=“GI:4557369”

TABLE 2

| | nucleotide | nucleotide | | |
|---|---|---|---|---|
| | position | position | | |
| nucleotide | relative to | relative to | position in | amino acid |
| change | SEQ. ID NO 1 | SEQ. AL035668 | gene | change |
| A to G | −2047 | 116454 | promoter | |
| T to C | −1136 | 117365 | promoter | |
| (ATTT)n | −901 | 117600 | promoter | |
| C to T | −638 | 117863 | promoter | |
| C to T | −568 | 117933 | promoter | |
| T to C | −72 | 118429 | promoter | |
| G to A | 70 | 118570 | promoter | |
| A | 368 | 118868 | promoter | |
| insertion | | | | |
| A to G | 420 | 118920 | promoter | |
| A to G | 472 | 118972 | promoter | |
| G to C | 1464 | 119964 | 5′ utr | |
| G to A | 1722 | 120222 | 5′ utr | |
| C to G | 1914 | 120414 | 5′ utr | |
| A to C | 2536* | 121036 | intron 1 | |
| C to T | 2866 | 121366 | intron 1 | |
| G to T | 3145 | 121645 | intron 1 | |
| T to G | 3747 | 122247 | exon 2 | serine to |
| | | | | alanine |
| A to G | 3899* | 122399 | exon 2 | |
| G to T | 3918 | 122418 | exon 2 | alanine to |
| | | | | serine |
| A to G | 4181 | 122681 | intron 2 | |
| G to A | 4244 | 122744 | intron 2 | |
| A to T | 4359 | 122859 | intron 2 | |
| G to A | 4435 | 122935 | intron 2 | |
| T | 4712 | 123212 | intron 2 | |
| insertion | | | | |
| T to A | 5041 | 123541 | intron 2 | |
| C to T | 5048 | 123548 | intron 2 | |
| G to A | 5787 | 124287 | intron 2 | |
| G to A | 6217 | 124717 | intron 2 | |
| G to A | 7111* | 125611 | intron 2 | |
| A to T | 7162 | 125662 | intron 2 | |
| T to C | 7781* | 126281 | intron 2 | |
| A to G | 7828 | 126328 | intron 2 | |
| C to T | 7874 | 126374 | intron 2 | |
| G to C | 8035* | 126535 | intron 2 | |
| A to C | 8083 | 126583 | intron 2 | |
| T to G | 8463 | 126963 | intron 2 | |
| G to A | 9013* | 127513 | intron 2 | |
| G to A | 9082 | 127582 | intron 2 | |
| G to T | 10631 | 129131 | intron 2 | |
| A to G | 10841 | 129341 | intron 2 | |
| A to T | 11980* | 130480 | exon 2 | arginine to |
| | | | | serine |
| C to T | 12571 | 131071 | exon 2 | |
| A to C | 12845* | 131345 | 3′ utr | |
| T to C | 13066 | 131566 | 3′ utr | |
| A to G | 13209* | 131709 | 3′ utr | |
| C to A | 13296* | 131796 | 3′ utr | |
| 4 bp | 13533-13536 | 132033-132036 | 3′ utr | |
| deletion | | | | |

*known in SNP databases

### Example 2

Direct Sequencing of the BMP2 Nucleic Acid Sequence Reveals Other Polymorphisms.

Additional genetic markers were identified in the BMP2 nucleic acid by direct sequencing of the region in different populations. These are listed in Table 3 with nucleotide position relative to SEQ. AL035668 as in earlier SNP update file.

TABLE 3

| | | nucleotide | nucleotide | | |
|---|---|---|---|---|---|
| deCODE | | position relative | position relative | | |
| numbering | type of change | to SEQ. ID NO 1 | to AL035668 | location | Public name |
| P4019 | C to T | | 112569 | | |
| P4204 | A to C | | 112754 | | |
| P4337 | T to G | | 112887 | | |
| P4617 | T to A | | 113167 | | |
| P4730 | A to G | | 113280 | | |
| P4765 | T to C | | 113315 | | |
| P4822 | A to G | | 113372 | | |
| P5831 | T to C | | 114381 | | |
| P6121 | A to C | | 114671 | | rs173106 |
| P6136 | A to T | | 114686 | | |
| P6784 | C to T | | 115334 | | rs969643 |
| P6854 | A to C | | 115404 | | |
| P7420 | G to A | | 115970 | | |
| P7904 | A to G | −2047 | 116454 | promoter | |
| P8815 | T to C | −1136 | 117365 | ″ | |
| P9050 | (ATTT)n | −901 | 117600 | ″ | |
| P9313 | C to T | −638 | 117863 | ″ | |
| P9383 | C to T | −568 | 117933 | ″ | |
| P9879 | T to C | −72 | 118429 | ″ | |
| B70 | G to A | 70 | 118570 | ″ | |
| B368 | A insertion | 368 | 118868 | ″ | |
| B420 | A to G | 420 | 118920 | ″ | |
| B472 | A to G | 472 | 118972 | ″ | |
| B1464 | G to C | 1464 | 119964 | 5′ utr | |
| B1722 | G to A | 1722 | 120222 | ″ | |
| B1914 | C to G | 1914 | 120414 | ″ | |
| B2536 | A to C | 2536 | 121036 | intron 1 | |
| B2866 | C to T | 2866 | 121366 | ″ | |
| B3145 | G to T | 3145 | 121645 | ″ | |
| B3747 | T to G | 3747 | 122247 | exon 2 | rs2273073 |
| B3899 | A to G | 3899 | 122399 | ″ | rs1049007 |
| B3918 | G to T | 3918 | 122418 | ″ | |
| B4181 | A to G | 4181 | 122681 | intron 2 | |
| B4244 | G to A | 4244 | 122744 | ″ | |
| B4359 | A to T | 4359 | 122859 | ″ | |
| B4435 | G to A | 4435 | 122935 | ″ | |
| B4712 | T insertion | 4712 | 123212 | ″ | |
| B5041 | T to A | 5041 | 123541 | ″ | |
| B5048 | C to T | 5048 | 123548 | ″ | |
| B5787 | G to A | 5787 | 124287 | ″ | |
| B6217 | G to A | 6217 | 124717 | ″ | |
| B7111 | G to A | 7111 | 125611 | ″ | rs235764 |
| B7262 | A to T | 7162 | 125662 | ″ | |
| B7781* | T to C | 7781 | 126281 | ″ | rs1875274 |
| B7828* | A to G | 7828 | 126328 | ″ | |
| B7874* | C to T | 7874 | 126374 | ″ | |
| B8035* | G to C | 8035 | 126535 | ″ | rs235766 |
| B8083 | A to C | 8083 | 126583 | ″ | |
| B8463 | T to G | 8463 | 126963 | ″ | rs235767 |
| B9013 | G to A | 9013 | 127513 | ″ | rs1005464 |
| B9082 | G to A | 9082 | 127582 | ″ | |
| B10631 | G to T | 10631 | 129131 | ″ | |
| B10841 | A to G | 10841 | 129341 | ″ | |
| B11980 | A to T | 11980 | 130480 | exon 2 | rs235768 |
| B12571 | C to T | 12571 | 131071 | ″ | |
| B12845 | A to C | 12845 | 131345 | 3′ utr | rs15705 |
| B13066 | T to C | 13066 | 131566 | ″ | rs3178250 |
| B13209 | A to G | 13209 | 131709 | ″ | rs235769 |
| B13296 | C to A | 13296 | 131796 | ″ | rs170986 |
| B13533del4 | 4 bp deletion | 13533-13536 | 132033 | ″ | |
| D841 | C to T | | 132877 | | |
| D873 | T to C | | 132909 | | |
| D1094 | T to C | | 133130 | | rs235770 |
| D1226 | A to C | | 133262 | | |
| D1354 | G to A | | 133390 | | |
| D1550 | C to T | | 133586 | | TSC0078312/ |
| | | | | | rs28488 |
| D1886 | A to G | | 133922 | | |
| D2048 | C to T | | 134084 | | rs235772 |
| D2269 | C to T | | 134305 | | |
| D2319 | T to A | | 134355 | | |
| D2568 | A to C | | 134604 | | |
| D5348 | C to T | | 137384 | | |
| D5449 | G to A | | 137485 | | |
| D5498 | C to T | | 137534 | | |
| D5643 | G to T | | 137679 | | |
| D6220 | A to G | | 138256 | | rs28151 |
| D6440 | A to G | | 138476 | | |
| D6448 | G to C | | 138484 | | |
| D6683 | C to T | | 138719 | | |
| D6971 | G to T | | 139007 | | TSC0191642/ |
| | | | | | rs910141 |
| D7006 | C to G | | 139042 | | |
| D7355 | C to G | | 139391 | | |
| D7630 | G to A | | 139666 | | |
| D8183 | C to T | | 140219 | | rs235750 |
| D8629 | T to C | | 140665 | | |
| D8632 | A to G | | 140668 | | |
| D8862 | G to A | | 140898 | | |
| D9005 | A to G | | 141041 | | |
| D9036 | C to T | | 141072 | | |
| D9043 | C to T | | 141079 | | |
| D9126 | G to A | | 141162 | | |
| D9206 | T to C | | 141242 | | rs235750 |
| D9473 | T to G | | 141509 | | |
| D9617 | C to T | | 141653 | | |
| D9970 | G to T | | 142006 | | rs235748 |
| D10019 | G to A | | 142055 | | |
| D10402 | T to C | | 142438 | | |
| D10540 | G to A | | 142576 | | |
| D10554 | T to C | | 142590 | | |
| D10699 | C to A | | 142735 | | |
| D11023 | T to C | | 143059 | | |
| D11373 | G to A | | 143409 | | |
| D11395 | A to G | | 143431 | | |
| D11592 | A to G | | 143628 | | |
| D12541 | C to T | | 144577 | | |
| D12645 | A to T | | 144681 | | |
| D12699 | G to A | | 144735 | | |
| D12908 | C to A | | 144944 | | |
| D13002 | T to C | | 145038 | | |
| D13071 | T to A | | 145107 | | |
| D13256 | G to A | | 145292 | | |
| D13259 | G to T | | 145295 | | |
| D13488 | G to A | | 145524 | | |
| D13749 | A to G | | 145785 | | |
| D14613 | T to C | | 146649 | | |
| D14664 | C to T | | 146700 | | |
| D14956 | G to A | | 146992 | | |
| D15562 | C to T | | 147598 | | |
| D15601 | T to C | | 147637 | | |
| D15827 | C to T | | 147863 | | |
| D16270 | A to G | | 148306 | | |
| D16345 | C to T | | 148381 | | |
| D16407 | T to C | | 148443 | | |
| D16595 | C to G | | 148631 | | |
| D17037 | T to C | | 149073 | | |
| D17242 | G to A | | 149278 | | |
| D17493 | A to G | | 149529 | | rs1116867 |
| D17684 | G to T | | 149720 | | |
| D17794 | G to A | | 149830 | | |
| D18035 | A to T | | 150071 | | |
| D18292 | C to A | | 150328 | | |
| D18307 | C to T | | 150343 | | |
| D18513 | C to G | | 150549 | | |
| D18641 | A to G | | 150677 | | |
| D18855 | A to T | | 150891 | | |
| D19047 | C to A | | 151083 | | |
| D19354 | G to A | | 151390 | | |
| D19690 | G to A | | 151726 | | |
| D20383 | A to G | | 152419 | | |
| D20945 | T to A | | 152981 | | |
| D20958 | C to T | | 152994 | | |
| D20961 | C to T | | 152996 | | |
| D21101 | C to T | | 153137 | | |
| D21190 | C to A | | 153226 | | |
| D21354 | G to A | | 153390 | | |
| D21382 | T to C | | 153418 | | |
| D22041 | A to G | | 154077 | | TSC0278787 |
| D22254 | C to G | | 154290 | | TSC0278788 |
| D22326 | C to T | | 154362 | | |
| D22530del6 | del6bp | | 154566 | | |
| D22603 | T to C | | 154639 | | |
| D22641 | C to T | | 154677 | | |
| D22641 | C to T | | 154677 | | |
| D23348 | C to T | | 155384 | | |
| D24843 | G to A | | 156879 | | |
| D25216 | A to C | | 157252 | | |
| D25494 | C to T | | 157530 | | |
| D25528 | T to C | | 157564 | | rs2876039 |
| D25715 | A to G | | 157751 | | |
| D26836 | A to C | | 158872 | | |
| D28047 | G to A | | 160083 | | |
| D28047 | G to A | | 160083 | | |
| D28783 | C to T | | 160819 | | |
| D29019 | G to A | | 161055 | | |
| D29281 | A to C | | 161317 | | |
| D29461 | T to C | | 161497 | | |
| D29569 | C to T | | 161605 | | |
| D30340 | C to T | | 162376 | | |
| D30630 | G to A | | 162666 | | |
| D31474 | G to T | | 163510 | | |
| D31616 | T to A | | 163652 | | |
| D32258 | T to C | | 164294 | | |
| D32371 | A to C | | 164407 | | |
| D33541 | T to C | | 165577 | | |
| D34249 | T to G | | 166285 | | |
| D34699 | A to G | | 166735 | | |
| D35273 | C to A | | 167309 | | |
| D35548 | C to T | | 167584 | | |
| D35650 | G to T | | 167686 | | TSC032068 |

Additional linkage data of various SNPs to osteoporosis phenotypes is listed in Table 4. Table 4 lists the p-value for the association of the SNP with the phenotype (p-value), the relative risk (r), percentage of individuals affected (Aff. freq. carrier), number of affected individuals (#aff), percentage of controls (Ctrl. freq. carrier), number of controls (#ctrl), identification of the allele, and phenotype screened.

TABLE 4
Association of SNPs with osteoporosis phenotypes.

| p-val | r | # aff | Aff. freq. carrier | # con | Ctrl. freq. carrier | allele | base | marker | Phenotype |
|---|---|---|---|---|---|---|---|---|---|
| 0.0171 | 2.1 | 648 | 6% | 404 | 3% | 2 | G | B3747 | Linkage phenotype |
| 0.0174 | 1.4 | 832 | 13% | 1211 | 10% | 2 | G | B420 | Linkage phenotype |
| 0.0443 | 1.2 | 342 | 62% | 428 | 56% | 2 | G | D6440 | Linkage phenotype |
| 0.0477 | 1.4 | 426 | 16% | 577 | 12% | 3 | T | D35548 | Linkage phenotype |
| 0.0498 | 1.3 | 570 | 36% | 401 | 30% | 3 | T | TSC0428253 | Linkage phenotype |
| 0.0254 | 1.4 | 171 | 63% | 356 | 54% | 2 | G | TSC0293456 | fracture |

While this invention has been particularly shown and described with reference to preferred embodiments thereof, it will be understood by those skilled in the art that various changes in form and details can be made therein without departing from the spirit and scope of the invention as defined by the appended claims.

What is claimed is:

1. A method of diagnosing a susceptibility to osteoporosis in an individual, comprising detecting at least one polymorphism in a human BMP2 gene of SEQ ID NO: 1, wherein the presence of a “G” at nucleotide position 122247 of AL035668; the presence of a “G” allele at position 118920 of AL035668; the presence of a “T” allele at position 167584 of AL035668; the presence of a “G” allele at position 138476 of AL035668; the presence of a “T” allele at position 167584 of AL035668; the presence of a “T” allele at TSC0428253; or the presence of the “G” allele of TSC0293456 is indicative of a susceptibility to osteoporosis, compared with an individual having a “T” at nucleotide position 122247 of AL035668; an “A” allele at position 118920 of AL035668; a “C” allele at position 167584 of AL035668; an “A” allele at position 138476 of AL035668; a “C” allele at position 167584 of AL035668; a “C” allele at TSC0428253; or the presence of the “A” allele of TSC0293456.

2. The method of claim 1, wherein the polymorphism is detected in a sample from a source selected from the group consisting of: blood, serum, cells and tissue.

3. A method of diagnosing a susceptibility to osteoporosis in an individual, comprising detecting at least one polymorphism in a human BMP2 gene of SEQ ID NO: 1, wherein the polymorphism is selected from the group consisting of T to G at position 122247 of AL035668; A to G at position 118920 of AL035668; C to T at position 167584 of AL035668; A to G at position 138476 of AL035668; C to T at position 167584 of AL035668; C to T at TSC0428253; A to G at TSC0293456 and combinations thereof.

4. The method of claim 3, wherein the polymorphism is detected in a sample from a source selected from the group consisting of: blood, serum, cells and tissue.

5. An isolated nucleic acid molecule comprising the nucleic acid having SEQ ID NO: 1 with one or more of the nucleic acid changes selected from the group consisting of: T to G at position 122247 of AL035668; C to T at position 121366 of AL035668; A to G at position 118920 of AL035668; C to T at position 167584 of AL035668; A to G at position 138476 of AL035668 and combinations thereof.

6. An isolated polypeptide comprising an amino acid sequence selected from the group consisting of: an alanine at amino acid position 37, a serine at amino acid position 94, a serine at amino acid position 189, or combinations thereof.

7. A method of diagnosing a susceptibility to osteoporosis, comprising detecting a polypeptide according to claim 6 that is indicative of a susceptibility to osteoporosis.

8. The method of claim 7, wherein the determination of an amino acid at a position selected from the group consisting of: position 37, position 94, position 1891 and combinations thereof, comprises contacting the sample with an antibody specific for either the reference amino acid or the variant amino acid.

9. A pharmaceutical composition comprising a polypeptide of claim 6.

10. A method of treating osteoporosis in an individual, comprising administering to the individual, isolated polypeptide of claim 6, in a therapeutically effective amount.

11. A kit comprising:
a) at least one antibody selected from the group consisting of: an antibody specific for the BMP2 protein comprising a serine at amino acid position 37, an alanine at amino acid position 37, an alanine at amino acid position 94, a serine at amino acid position 94, an arginine at amino acid position 189, a serine at amino acid position 189, or combinations thereof; and
b) a reference BMP2 protein sample.
